(19) United States
(12) Patent Application Publication
(10) Pub. No.: US 2024/0165270 A1
(43) Pub. Date: May 23, 2024

(54) MeCP2 EXPRESSION CASSETTES

(71) Applicants: THE UNIVERSITY COURT OF THE UNIVERSITY OF EDINBURGH, Edinburgh (GB); THE UNIVERSITY COURT OF THE UNIVERSITY OF GLASGOW, Glasgow (GB)

(72) Inventors: Adrian BIRD, Edinburgh (GB); Rebekah TILLOTSON, Edinburgh (GB); Stuart Robert COBB, Edinburgh (GB); Ralph David HECTOR, Edinburgh (GB)

(21) Appl. No.: 18/503,439

(22) Filed: Nov. 7, 2023

Related U.S. Application Data
(62) Division of application No. 16/497,277, filed on Sep. 24, 2019.

(30) Foreign Application Priority Data
Mar. 24, 2017 (GB) 1704704.4
Mar. 24, 2017 (GB) 1704722.6

(51) Int. Cl. A61K 48/00; A61K 9/00; A61P 25/14; C07K 14/00; C07K 14/47; C12N 7/00; C12N 15/86

(52) U.S. Cl. CPC A61K 48/0066; A61K 9/0019; A61K 9/0085; A61P 25/14; C07K 14/001; C07K 14/4703; C12N 7/00; C12N 15/86; A61K 38/00; C12N 2750/14143

(57) ABSTRACT

MECP2 EXPRESSION CASSETTES. The present invention provides nucleic acid molecules comprising a MeCP2 expression cassette, the expression cassette comprising, in operable linkage from 5′ to 3′: a 5′ transcriptional control region comprising a promoter capable of driving transcription in neural cells; an open reading frame encoding a MeCP2 protein; translation control signals; a 3′ untranslated region (3′UTR) comprising one or more of: (i) a binding site for mir-22; (ii) a binding site for mir-19; (iii) a binding site for miR-132; (iv) a binding site for miR124; and (v) an AU-rich element; and transcriptional termination signals; wherein the MeCP2 expression cassette is not more than about 5 kb in length. The invention further provides viral vectors, especially vectors derived from adeno-associated virus (AAV), for use in therapeutic delivery of such expression cassettes. The nucleic acid molecules and viral vectors disclosed herein provide novel tools for expressing MeCP2 and are of particular value in the treatment of disorders associated with reduced MeCP2 activity, including Rett syndrome.

This application is a divisional of U.S. patent application Ser. No. 16/497,277, filed Sep. 24, 2019, which is a National Stage Entry of PCT/GB2018/050773, filed Mar. 23, 2018, which claims the benefit of Great Britain Application No. 1704704.4, filed Mar. 24, 2017, and Great Britain Application No. 1704722.6, filed Mar. 24, 2017, which are hereby incorporated by reference in their entirety.

### SEQUENCE LISTING

The instant application contains a Sequence Listing which is being submitted electronically in XML format and is hereby incorporated by reference in its entirety. Said XML copy, created on Oct. 23, 2023, is named 136526.001202.xml and is 59,626 bytes in size.

### FIELD OF THE INVENTION

The invention relates to an expression cassette comprising a gene encoding MeCP2 protein, and to viral vectors, especially vectors derived from adeno-associated virus (AAV), for use in therapeutic delivery of such expression cassettes.

### BACKGROUND TO THE INVENTION

Rett syndrome (RTT; OMIM 312750) is a neurological disorder characterized by a constellation of clinical diagnostic and associated features and with overt onset occurring several months postnatally¹. Typical RTT is almost exclusively caused by de novo germline mutations in the X-linked gene, MECP2²; reviewed in^{3, 4}. Several mouse models of RTT have been generated that harbour Mecp2 deletions⁵⁻⁷ or knocked-in mutations⁸⁻¹¹. Many of these models recapitulate the principal features that characterize RTT in humans, although there are differences that reflect the phenotypic variability seen in patients¹²⁻¹⁴. Despite the severity of RTT-like phenotypes, genetic reactivation of silenced Mecp2 in conditional knockout mice resulted in a robust and enduring reversal of phenotypes¹⁵⁻¹⁷.

This inherent reversibility of the phenotype, added to the lack of obvious targets for pharmacotherapy, makes gene therapy an obvious candidate therapeutic strategy in RTT. However, there are significant challenges to a gene transfer approach, including the requirement to transduce sufficient numbers of neurons in the brain¹⁶ and the avoidance of deleterious overexpression¹⁸.

Previous attempts at MECP2 gene transfer using AAV9 vectors were confounded by limited brain transduction efficiency and toxicity^{19, 20} while efficacy in other studies using self-complementary AAV (scAAV)²¹ may have been compromised by use of a construct exceeding the packaging capacity of the vector.

### SUMMARY OF THE INVENTION

Despite the progress made in recent years in establishing the suitability of Rett syndrome for treatment by gene transfer, existing vectors pose efficacy and safety concerns. Low levels of transduction or gene expression may not successfully rescue the affected phenotype, while over-expression results in toxicity. Consequently there remains a need for vectors for delivery of MeCP2 which have improved efficacy and/or safety profiles compared to currently available options.

Vectors based on adeno-associated virus (AAV) are promising candidates for gene delivery but have limited capacity, being capable of carrying a genome of about 4.7 kb to about 5 kb of single stranded DNA. An AAV vector genome possesses a 145 nucleotide palindromic repeat sequence at each end (also known as an inverted terminal repeat, or ITR), reducing the capacity of transgene payload to about 4.4 kb to about 4.7 kb.

The human MECP2 gene is large, having a very long 3′ untranslated region of 8.5 kb in addition to the promoter and coding sequences. It is therefore impossible to include the entire gene, with all of its endogenous upstream and downstream regulatory sequences, in a recombinant AAV (rAAV) vector.

So-called “self complementary AAVs” (scAAVs) can provide more efficient transgene expression as compared to conventional rAAV vectors. However, an scAAV vector genome contains two copies of the same transgene payload in opposite orientations, and so in practice has only half the coding capacity of an rAAV vector, further exacerbating the difficulty.

The present invention provides a nucleic acid molecule comprising a MeCP2 expression cassette, the expression cassette comprising, in operable linkage from 5′ to 3′:
a 5′ transcriptional control region comprising a promoter capable of driving transcription in neural cells;
an open reading frame encoding a MeCP2 protein;
translation control signals;
a 3′ untranslated region (3′UTR) comprising one or more of:
   (i) a binding site for mir-22;
   (ii) a binding site for mir-19;
   (iii) a binding site for miR-132;
   (iv) a binding site for miR124; and
   (v) an AU-rich element; and
transcriptional termination signals;
   wherein the MeCP2 expression cassette is not more than about 5 kb in length.

The nucleic acid may be linear or circular, and single or double stranded. For example, the nucleic acid may be a plasmid or other expression vector, including a viral vector. Although much of the following discussion concentrates on AAV vectors, it will be understood that the expression cassette may also be employed in the context of other viral vectors, including adenoviral vectors and retroviral (e.g. lentiviral) vectors.

In some embodiments, e.g. when the expression cassette is for incorporation into a rAAV vector, the expression cassette may be not more than about 4.9 kb, 4.8 kb, 4.7 kb, 4.6 kb, 4.5 kb or 4.4 kb in length. Preferably it is not more than 4.4. kb in length.

The nucleic acid molecule may further comprise ITR sequences. Thus the nucleic acid molecule may comprise a 5′ ITR and a 3′ ITR, wherein the ITRs flank the expression cassette

The nucleic acid molecule may be a rAAV genome. Thus the invention further provides a rAAV genome comprising a 5′ ITR, a MeCP2 expression cassette of the invention, and a 3′ ITR.

In other embodiments, e.g. when the expression cassette is for incorporation into a scAAV vector, the expression cassette may be not more than about 2.4 kb, not more than 2.3 kb, or not more than 2.2 kb in length. Preferably it is not more than 2.2 kb in length.

An scAAV vector genome comprises inverted repeats of the payload sequence located between the ITRs. Thus it is possible for the vector genome molecule to adopt a hairpin-like structure in which the two complementary payload sequences hybridise to one another intramolecularly, or for two copies of the full-length genome to hybridise to one another via the payload sequences. (The ITR sequences will not necessarily hybridise to one another, because the ITRs at each end may not have precisely complementary sequences, and also because each ITR is likely to form its own internal secondary structure.)

Thus the invention further provides a nucleic acid molecule comprising, from 5′ to 3′, a MeCP2 expression cassette of the invention and the reverse complement of said expression cassette.

The nucleic acid molecule may further comprise ITR sequences. Thus the nucleic acid molecule may comprise a 5′ ITR, a MeCP2 expression cassette of the invention, the reverse complement of said expression cassette, and a 3′ ITR. The nucleic acid molecule may be a scAAV vector genome.

The ITR sequences may be from any suitable AAV type. For example, they may be from AAV2.

An AAV vector may have genomic ITRs from a first serotype (“A”) and proteins from a second serotype (“B”). Such a vector may be referred to as type “AAV A/B”. However, since the viral proteins largely determine the serological properties of the virion particle, such a vector may still be referred to as being of serotype B.

The 5′ transcription regulatory region may comprise one, two or all three of the core MeCP2 promoter, the MeCP2 silencer element, and a CNS regulatory element.

The 3′UTR typically comprises a binding site for one or more of miR-22, miR-19, miR-132 and miR-124. For example, it may contain binding sites for at least 2, at least 3, or all 4 of miR-22, miR-19, miR-132 and miR-124.

For example, the 3′UTR may contain binding sites for:
miR-22 and miR-19;
miR-22 and mir-132;
miR-22 and miR124;
miR-19 and miR-132;
miR-19 and miR-124;
miR-132 and miR-124;
miR-22, miR-19 and miR-132;
miR-22, miR-19 and miR-124;
miR-22, miR-132 and miR-124;
miR-19, miR-132 and miR-124;
or miR-22, miR-19, miR-132 and miR-124.

Additionally or alternatively, the 3′UTR may comprise an AU-rich element. Thus it may contain an AU-rich element alone, or in combination with binding sites for one or more of one of miR-22, miR-19, miR-132 and miR-124. For example, the 3′UTR may contain binding sites for at least 2, at least 3, or all 4 of miR-22, miR-19, miR-132 and miR-124 in combination with an AU-rich element.

For example, the 3′UTR may contain an AU-rich element in combination with binding sites for:
miR-22 and miR-19;
miR-22 and mir-132;
miR-22 and miR124;
miR-19 and miR-132;
miR-19 and miR-124;
miR-132 and miR-124;
miR-22, miR-19 and miR-132;
miR-22, miR-19 and miR-124;
miR-22, miR-132 and miR-124;
miR-19, miR-132 and miR-124;
or miR-22, miR-19, miR-132 and miR-124.

The AU-rich element and the miRNA binding sites regulate mRNA stability and/or expression from the mRNA. These elements may be present in any order. However, it may be desirable that those elements which are present occur in the order miR-22 site, miR-19 site, miR-132 site, AU-rich element and miR-124 site, from 5′ to 3′ of the sense strand.

The invention further provides an AAV virion particle comprising a nucleic acid molecule or AAV genome as described. The AAV genome may be a rAAV genome or a scAAV genome.

The virion particle may be regarded as a gene delivery vehicle for delivering nucleic acid encoding MeCP2 protein to a target cell, and capable of inducing expression of MeCP2 protein in a target cell.

The AAV virion may be of any suitable serotype. Serotypes AAV9 and AAV PHP.B may be particularly preferred due to their capacity for transduction of neural cells.

The invention further provides a cell comprising a nucleic acid as described herein. The cell may, for example, be a packaging cell, capable of producing a virion particle as described.

The cell will be capable of expressing AAV proteins (e.g. rep and cap proteins) and of supporting assembly and release of infectious AAV virion particles as described. It may also possess helper virus functions, e.g. from adenovirus, El-deleted adenovirus or herpesvirus.

The invention further provides a nucleic acid or AAV virion particle as described herein for use in enhancing expression of MeCP2 protein in a target cell.

The invention further provides a nucleic acid or AAV virion particle as described herein for use in the treatment of Rett syndrome.

The invention further provides a pharmaceutical composition comprising a nucleic acid or AAV virion of the invention, optionally in combination with a pharmaceutically acceptable carrier.

The invention further provides a method of treatment of Rett syndrome in an subject in need thereof, comprising administering a nucleic acid or AAV virion particle as described herein to the subject. Administration may be via any suitable peripheral or central route, but intravenous and intrathecal administration may be particularly suitable.

The invention further provides the use of a nucleic acid or AAV virion particle in the preparation of a medicament for the treatment of Rett syndrome.

Thus the subject to whom the nucleic acid or virion is to be administered may already be affected by Rett syndrome, or may be at risk of developing Rett syndrome. The subject may have been identified as being affected by or at risk of developing Rett syndrome, e.g. by means of genetic testing, e.g. for one or more mutations (especially loss of function mutations) in the MECP2 gene.

Thus the invention provides a method comprising the step of testing a subject for the presence of one or more mutations in the MECP2 gene indicative of the presence of, or a predisposition to, Rett syndrome, and selecting the subject for treatment with a nucleic acid or AAV virion as described herein if one or more such mutations is identified.

Where a “nucleic acid molecule” is referred to in this specification, it may be RNA or DNA, and single or double stranded, unless the context requires otherwise.

An AAV genome molecule is necessarily a single stranded DNA molecule. Although an scAAV genome has the capacity to adopt a hairpin secondary structure, a single scAAV genome will generally be regarded herein as a single stranded molecule since it still consists only of a single continuous strand of DNA. A complex of two scAAV genomes hybridised to one another could be considered to be a double stranded molecule.

Reference is made in this specification to both DNA and RNA sequences. It will be apparent to the reader that sequences containing T refer to DNA molecules, such as AAV genome sequences or expression constructs, while sequences containing U refer to RNA molecules such as mRNA transcripts derived by transcription from DNA expression cassettes, e.g. in AAV genomes.

### DESCRIPTION OF THE DRAWINGS

FIG. 1. Systemic delivery of the 1^{st} generation vector to Mecp2^{-/y} mice revealed therapeutic efficacy and a narrow therapeutic window.

(a) Kaplan-Meier survival plot for Mecp2^{-/y} mice injected with different doses [1×10¹¹ (n=10), 1×10¹² (n=8) and 1×10¹³ (n=5) vg/mouse] of 1^{st} generation vector compared to vehicle-treated animals (WT; n=9, Mecp2^{-/y}; n=16). The median survival period in Mecp2^{-/y} mice treated with 1×10¹² vg/mouse was significantly higher than that in vehicle-treated controls (27.14 versus 11.64 weeks, p=0.001, Mantel-Cox test). (b-c) plots showing mean bodyweight and aggregate severity scores, respectively, for Mecp2^{-/y} mice treated with 1×10¹¹, 1×10¹² vg/mouse or vehicle. Arrows indicate age at injection; data presented as mean±SEM. (d) Dose-dependent transduction efficiency (Myc-positive nuclei as a proportion of DAPI-positive nuclei) across different brain regions. Data presented as mean±SEM (n=3 mice per group). CA1 indicates hippocampal region CA1.

FIG. 2. Intravenous injection of the 1^{st} generation vector resulted in pathological changes in the liver.

(a-d) Representative H&E-stained liver sections from wild-type mice injected with (a) vehicle or (b-d) different doses of vector. (e) Liver section from a mouse injected intravenously with a GFP control vector, counterstained with DAPI. (f) Representative H&E-stained liver section from a GFP vector-treated mouse. Arrows indicate mononuclear cell infiltration, vacuolation and/or loss of hepatocytes. Dashed white line indicates cellular swelling. Scale bar indicates 20 μm; CV indicates central vein.

FIG. 3. Improved survival and bodyweight of Mecp2^{T158M/Y} mice after systemic delivery of the 1^{st} generation vector.

(a) Survival plot for treated Mecp2^{T158M/y} mice. Arrow indicates age at injection. (b-c) plots of bodyweight and aggregate severity score, respectively, for Mecp2^{T158M/y} mice treated with 1×10¹² vg/mouse of 1^{st} generation vector and control groups (Mecp2^{T158M/y} and WT) treated with vehicle. Data presented as mean±SEM. (d) Transduction efficiency in the brain of treated mice (Myc-positive nuclei as a proportion of DAPI-positive nuclei; n=3 mice).

FIG. 4. Nuclear localisation of MeCP2 in untreated and treated Mecp2^{T158M/Y} mice.

Representative confocal images of the CA1 region of the hippocampus. (a) Endogenous MeCP2 exhibits heterochromatin-enriched localisation in wild-type nuclei, while GFP-tagged MeCP2 exhibits decreased heterochromatin localization (i.e. more diffuse labelling) in nuclei from Mecp2^{T158M/y} mice. (b) Images demonstrating heterochromatin-enriched localisation of exogenously-derived MeCP2 in nuclei of transduced cells in Mecp2^{T158M/y} mice treated with the 1^{st} generation vector. White arrows indicate transduced cells (Myc-positive). Scale bar indicates 20 μm.

FIG. 5. Therapeutic efficacy of 2^{nd} generation vector after systemic delivery to Mecp2^{-/y} mice.

(a) Design features of our 2^{nd} generation vector summarized (see text and Suppl. FIG. 7 for details). (b) Survival plot for Mecp2^{-/y} mice treated intravenously with 1×10¹² vg/mouse of the 2^{nd} generation vector (median survival=29.9 weeks) or an identical dose of 1^{st} generation vector (median survival=27.1 weeks) or vehicle (median survival=11.6 weeks). Arrow indicates age at injection. (c-d) Plots showing mean bodyweight and aggregate severity scores, respectively, of Mecp2^{-/y} mice treated as in (b).

FIG. 6. Reduced expression of exogenous MeCP2 in the livers of mice treated with 2nd generation vector (a) Flattened confocal stack images from livers of mice one month after being injected intravenously at 5 weeks of age with the 2^{nd} generation vector or 1^{st} generation vector at 1×10¹² vg/mouse; confocal settings were the same in each case. Tissues were immunolabelled with anti-Myc and DAPI nuclear stain. Arrows indicate transduced cells (Myc-positive) and arrowheads indicate non-transduced cells. (b) Transduction efficiencies in the liver for both vectors. (c) Quantification of cellular levels of exogenous MeCP2 measured as anti-Myc immunofluorescence in transduced cells in the liver (n=3 mice, 1400 transduced cells). Data presented as mean±SEM. (d) Frequency distribution of cellular levels of exogenous MeCP2 in the liver, measured as in (c). (e) Liver sections stained with H&E showing vacuolation of hepatocytes (arrows) and sites of mononuclear cell infiltration (dashed circles). CV indicates central vein. White scale bar indicates 20 μm. (f) Quantification of density of inflammatory foci in the livers of treated mice (n=3 per group). Data presented as mean±SEM. *p<0.05, **p<0.01, ***p<0.001.

FIG. 7. Direct brain delivery of 2^{nd} generation vector to neonatal Mecp2^{-/y} mice revealed therapeutic efficacy. (a) Experimental design. (b) Survival plot showing extended survival of neonatally treated Mecp2^{-/y} mice (median survival=38.6 weeks; p<0.0001, Mantel-Cox test) compared with vehicle-treated animals (median survival=12.4 weeks). (c-d) Plots showing mean bodyweight and aggregate severity scores, respectively, for the mice shown in (b). (e) Representative confocal images from the cortex of injected wild-type mice. White arrows indicate transduced cells; arrowheads indicate non-transduced cells; scale bar indicates 20 μm. (f) Graph showing transduction efficiency in different brain regions (n=3 mice). (g) Frequency distribution of MeCP2 levels in transduced and non-transduced (‘native’) cells in the mouse cortex (n=3 mice; 954 transduced cells) data presented as mean±SEM.

FIG. 8. Expression of exogenous MeCP2 in the brain after intravenous injection of the 1^{st} generation vector. Representative confocal micrographs showing transgene expression in the hippocampal CA1 region in Mecp2^{-/y} mice treated intravenously with 1×10^{11, 1}×10¹² and 1×10¹³ vg/mouse of the 1^{st} generation vector (as revealed by anti-Myc tag immunolabelling). Arrows denote transduced cells and the lower panel shows co-localisation with DAPI. Scale bar=20 μm

FIG. 9. Systemic delivery of the 1^{st} generation vector to wild-type mice is tolerated at low doses but toxic at high doses.

(a) Survival plot showing the early toxicity observed after IV injection of a 1×10¹³ vg/mouse dose of the 1^{st} generation vector (green) compared to other doses and vehicle control. Arrow indicates age at injection. (b-c) Plots showing mean bodyweight and aggregate severity score, respectively, for these cohorts after injection. Data presented as mean±SEM. (d) Flattened confocal stack images of the hippocampus CA1 region of wild-type mice injected with 1×10¹³ vg/mouse of the 1^{st} generation vector. Tissues were immunolabelled with anti-Myc and anti-MeCP2 antibodies. White arrows indicate transduced cells. Scale bar indicates 20 μm. (e) Quantification of cellular levels of native MeCP2 and exogenous MeCP2 in transduced and non-transduced cells in the hippocampus CA1 region of wild-type mice (n=2 mice; 131 transduced cells and 172 non-transduced cells). Data presented as mean±SEM and normalised to native MeCP2. (f) Frequency distribution of normalised MeCP2 level in transduced and non-transduced cells. # indicates lethality at high dose.

FIG. 10. Intravenous injection of 1^{st} generation vector resulted in high level of exogenous MeCP2 expression in the liver.

(a) Representative confocal images of liver taken from WT mice injected intravenously with 1^{st} generation vector at the dose of 1×10¹³ vg/mouse. Sections were immunolabelled with anti-Myc (green), anti-MeCP2 (red) and DAPI nuclear stain (blue). White arrows indicate transduced cells, whereas yellow arrows indicate non-transduced cells. (b) Flattened confocal stack images taken from the CA1 region of the hippocampus (top) and from the liver (mice were injected intravenously with 1×10¹³ vg/mouse) using the same confocal settings. Arrows indicate nuclei with a high level of exogenous MeCP2 expression (based on fluorescence intensity of the anti-Myc antibody) and arrowheads indicate nuclei with low expression levels. Scale bar in (a) & (b)=20 μm. (c) measurement of the integrated pixel intensity per nucleus in liver (55 transduced cells and CA1 (131 transduced cells) of the same mice (n=3 mice). Data presented as mean±SEM.

FIG. 11. Comparison of Mecp2^{T158M/y} and Mecp2^{-/y} mice. (a) Survival plot for Mecp2^{T158M/y} mice (n=15) and Mecp2^{-/y} mice (n=29). (b-c) Plots showing no significant differences in mean bodyweight and aggregate severity score, respectively, between Mecp2^{T158M/y} and Mecp2^{-/y} mice. Data presented as mean±SEM.

FIG. 12. Novel vector design features, efficacy and liver phenotype.

(a) A summary of the design differences for three of the novel vectors described in the text. (b) Efficacy of these three novel vectors after intravenous injection of 1×10¹² vg/mouse to 4-5 weeks old Mecp2^{-/y} mice, expressed as increase in median survival relative to the vehicle controls (left; compared using Mantel-Cox test) and mean bodyweight at the age of 11 weeks (right) relative to the vehicle controls (one-way ANOVA with Tukey's post-hoc pairwise comparisons). *p<0.05, **p<0.01, ***p<0.001. (c) Representative H&E-stained liver sections from mice injected with JeT, 9.47 or spA vectors. Arrows indicate vacuolation of hepatocytes; scale bar indicates 20 μm.

FIG. 13. Design of the 2^{nd} generation vector construct.

Putative regulatory elements (RE) in the extended mMeP426 promoter and endogenous distal 3′-UTR are indicated. The extent of the mMeP229 promoter (used in the 1ₛₜ generation vector) is shown relative to mMeP426. The RDH1pA 3′-UTR consists of several exogenous microRNA (miR) binding sites incorporated as a ‘binding panel’ adjacent to a portion of the distal endogenous MECP2 polyadenylation signal and its accompanying regulatory elements. References with an asterisk indicate human in vitro studies, not rodent.

FIG. 14. Annotated sequence of 2n d generation expression cassette (SEQ ID NO: 32 and SEQ ID NO: 33).

FIG. 15. Schematic diagram of plasmid encoding the second generation MeCP2 vector.

FIG. 16. Full sequence (SEQ ID NO: 32) of the plasmid shown in FIG. 15. The expression cassette illustrated in FIG. 14 is single-underlined and the ITRs are double-underlined.

FIG. 17. Direct brain delivery of 2^{nd} generation vector to neonatal Mecp2^{-/y} mice revealed a similar therapeutic efficacy of both full-length MeCP2 and ΔNIC protein.

(a) Experimental design. (b) Plot showing mean aggregate severity scores of neonatally treated Mecp2^{-/y} mice (full-length MeCP2 versus ΔNIC protein (truncated MeCP2) compared with vehicle-treated animals. Wild-type mice injected with vector were indistinguishable from vehicle treated mice.

### DETAILED DESCRIPTION OF THE INVENTION

### Rett Syndrome

Rett syndrome (RTT) is a neurological disorder almost exclusively caused by de novo germline mutations in the X-linked gene, MECP2¹⁻⁴. It is characterized by a constellation of clinical diagnostic and associated features, with symptoms typically becoming overt only 6-18 months postnatally. The phenotype appears to be inherently reversible, as genetic reactivation of silenced Mecp2 in conditional knockout mice results in a robust and enduring reversal of symptoms. However, there are significant challenges to a gene transfer approach, including the requirement to transduce sufficient numbers of neurons in the brain¹⁶ and the avoidance of deleterious overexpression¹⁸.

The present vectors provide viable candidates for treatment of affected individuals, and may even offer the prospect of preventing development of detectable phenotype, if administered to an individual carrying a mutation in the MECP2 gene before symptoms become detectable. Thus treatment may be considered “therapeutic” or “prophylactic”. The term “therapy” will be used to refer to inhibition or reversal of established symptoms or phenotype, while “prophylaxis” will be used to refer to inhibiting or preventing development of symptoms in individuals not already displaying overt symptoms. Such individuals will typically have been identified early in life as carrying a loss of function mutation in the MECP2 gene, e.g. by appropriate genetic testing performed before 18 months post partum, e.g. before 12 months or before 6 months post partum.

### AAV Vectors

Adeno-associated virus (AAV) is a replication-deficient parvovirus, the single stranded DNA genome of which is about 4.7 kb in length including 145 nucleotide inverted terminal repeat (ITRs). The nucleotide sequence of the AAV serotype 2 (AAV2) genome is presented in Srivastava el al., J Virol, 45: 555-564 (1983) as corrected by Ruffing el al., J Gen Virol, 75: 3385-3392 (1994). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p i9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins.

As the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as an expression cassette, with the rep and cap proteins provided in trans. The sequence located between ITRs of an AAV vector genome is referred to herein as the “payload”.

The actual capacity of any particular AAV particle may vary depending on the viral proteins employed. Typically, the vector genome (including ITRs) is not more than about 5 kb, e.g. not more than about 4.9 kb, 4.8 kb or 4.7 kb.

The ITRs are each 145 bases in length. Thus, the payload is typically not more than about 4.7 kb, 4.6 kb, 4.5 kb or 4.4 kb in length. Preferably it is not more than 4.4. kb in length. A recombinant AAV (rAAV) may therefore contain up to about 4.7 kb, 4.6 kb, 4.5 kb or 4.4 kb of unique payload sequence.

However, following infection of a target cell, protein expression and replication from the vector requires synthesis of a complementary DNA strand to form a double stranded genome. This second strand synthesis represents a rate limiting step in transgene expression. The requirement for second strand synthesis can be avoided using so-called “self complementary AAV” (scAAV) vectors in which the payload contains two copies of the same transgene payload in opposite orientations to one another, i.e. a first payload sequence followed by the reverse complement of that sequence. These scAAV genomes are capable of adopting either a hairpin structure, in which the complementary payload sequences hybridise intramolecularly with each other, or a double stranded complex of two genome molecules hybridised to one another. Transgene expression from such scAAVs is much more efficient than from conventional rAAVs, but the effective payload capacity of the vector genome is halved because of the need for the genome to carry two complementary copies of the payload sequence.

An scAAV vector genome may contain one or more mutations in one of the ITR sequences to inhibit resolution at one terminal repeat, and consequently increase yield in an scAAV preparation. Thus one of the ITRs in an scAAV may be deleted for the terminal resolution site or may contain an inactivating mutation in the terminal resolution site. See, for example, Wang et al., Gene Therapy (2003) 10, 2105-2111 and McCarty et al., Gene Therapy (2003) 10, 2112-2118. It will therefore be apparent that the two ITR sequences at either end of an AAV genome need not be identical.

scAAVs are reviewed in McCarty, Molecular Therapy, 16(10), 2008, 1648-1656.

In this specification, the term “rAAV vector” is generally used to refer to vectors having only one copy of any given payload sequence (i.e. a rAAV vector is not an scAAV vector), and the term “AAV vector” is used to encompass both rAAV and scAAV vectors.

AAV sequences in the AAV vector genomes (e.g. ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11 and AAV PHP.B. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); AAV PHP.B is described by Deverman et al., Nature Biotech. 34(2), 204-209 and its sequence deposited under GenBank Accession No. KU056473.1.

It may be desirable to employ AAV-2 ITRs. The scAAV vectors described in the examples below contain AAV-2 ITRs having the sequences (SEQ ID NO: 1):

GCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGA
GTGG

### And (SEQ ID NO:2):

CCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGC
GCGC

Likewise the proteins present in AAV virion particles of the invention may be derived from any suitable AAV serotype. In general, the vectors are targeted to neural cells, although an ability to transduce glial cells may also be desirable. AAV-9 and AAV PHP.B may be particularly effective at transducing such cell types and so virion proteins, especially capsid (cap) proteins, from AAV-9 or AAV PHP.B may be particularly preferred. The capsid proteins may be pseudotyped to increase specificity or transduction efficiency of the target cell type. These AAV types are also capable of crossing the blood brain barrier, so are particularly appropriate if peripheral administration is required.

Virion particles comprising vector genomes of the invention are typically generated in packaging cells capable of replicating viral genomes, expressing viral proteins (e.g. rep and cap proteins), and assembling virion particles. Packaging cells may also require helper virus functions, e.g. from adenovirus, El-deleted adenovirus or herpesvirus. Techniques to produce AAV vector particles in packaging cells are standard in the art. Production of pseudotyped AAV is disclosed in, for example, WO 01/83692. In various embodiments, AAV capsid proteins may be modified to enhance delivery of the recombinant vector. Modifications to capsid proteins are generally known in the art. See, for example, US 2005/0053922 and US 2009/0202490.

One method of generating a packaging cell is to create a cell line that stably expresses all the necessary components for AAV particle production. For example, a plasmid (or multiple plasmids) comprising an AAV genome lacking AAV rep and cap genes, AAV rep and cap genes separate from the AAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of AAV. Other examples of suitable methods employ adenovirus or baculovirus rather than plasmids to introduce AAV genomes and/or rep and cap genes into packaging cells.

Alternatively, a packaging cell can be generated by simply transforming a suitable cell with one or more plasmids encoding an AAV genome, AAV proteins, and any required helper virus functions. The so-called “triple transfection” method utilises three plasmids each carrying one of these sets of genes. See Grieger et al., Nature Protocols 1(3), 1412-128 (2006) and references cited therein.

General principles of AAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

Techniques for scAAV production are described by Grieger et al., Molecular Therapy 24(2), 287-297, 2016.

The invention thus provides packaging cells that produce infectious AAV virion particles of the invention. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

### MeCP2 Protein

The vectors described in this specification carry an expression cassette encoding methyl CpG binding protein (MeCP2), which is an transcriptional regulator encoded on the X chromosome and highly expressed in neurons, especially in neurons in the brain and CNS. The terms MECP2 and Mecp2 are typically used to refer to the human and murine genes respectively. Although the vectors of the invention are envisaged for use primarily in humans, they may be employed in other species, especially in mouse and other animal models of Rett syndrome. Thus, the terms MECP2 and MeCP2 will be used to refer to genes and proteins from any appropriate species and should not be interpreted as being species-specific unless the context demands.

As discussed above, loss of function mutations in the MECP2 gene are implicated in development of Rett syndrome, primarily in females.

A MeCP2 protein is capable of inducing an increase in survival, an increase in body weight, and/or an increase in RTT-like aggregate severity score in juvenile male MeCP2−/y mice. See Guy et al, Reversal of neurological symptoms in a mouse model of Rett syndrome; Science 315(5815): 1143-1147 (2007).

For the purposes of assessing activity, administration may be via any appropriate route, e.g. via an AAV vector as described in this specification. Improvements are seen as compared to identical control mice given an otherwise identical control treatment lacking functional MeCP2 protein.

Without wishing to be bound by theory, a MeCP2 protein will typically be capable of binding to methylated DNA and of interacting with (e.g. binding to) components of the NCoR/SMRT co-repressor complex. Components of the co-repressor complex include NCoR, HDAC3, SIN3A, GPS2, SMRT, TBL1X and TBLR1. Thus a MeCP2 protein may be capable of recruiting components of the NCoR/SMRT co-repressor complex to methylated DNA.

There are two isoforms of human MeCP2 protein which differ in their N-terminal sequence.

Isoform 1 has the sequence (SEQ ID NO: 3):

MAAAAAAAPSGGGGGGEEERLEEKSEDQDLQGLKDKPLKFKKVKKDKKEE
KEGKHEPVQPSAHHSAEPAEAGKAETSEGSGSAPAVPEASASPKQRRSII
RDRGPMYDDPTLPEGWTRKLKQRKSGRSAGKYDVYLINPQGKAFRSKVEL
IAYFEKVGDTSLDPNDFDFTVTGRGSPSRREQKPPKKPKSPKAPGTGRGR
GRPKGSGTTRPKAATSEGVQVKRVLEKSPGKLLVKMPFQTSPGGKAEGGG
ATTSTQVMVIKRPGRK [symbol] PGSVVAAAAAEAKKKA
VKSVQETVLPIKKRKTRETVSIEVKEVVKPLLVSTLGEKSGKGLKTCKSP
GRKSKESSPKGRSSSASSPPKKEHHHHHHHSESPKAPVPLLPPLPPPPPE
PESSEDPTSPPEPQDLSSSVCKEEKMPRGGSLESDGCPKEPAKTQPAVAT
AATAAEKYKHRGEGERKDIVSSSMPRPNREEPVDSRTPVTERVSS

Isoform 2 has the sequence (SEQ ID NO: 4):

MVAGMLGLREEKSEDQDLQGLKDKPLKFKKVKKDKKEEKEGKHEPVQPSAHHSAEPAEAGKA
ETSEGSGSAPAVPEASASPKORRSIIRDRGPMYDDPTLPEGWTRKLKORKSGRSAGKYDVYL
INPQGKAFRSKVELIAYFEKVGDTSLDPNDEDFTVTGRGSPSRREQKPPKKPKSPKAPGTGR
GRGRPKGSGTTRPKAATSEGVQVKRVLEKSPGKLLVKMPFQTSPGGKAEGGGATTSTQVMVI

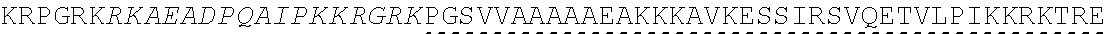

KRPGRKRKAEADPQAIPKKRGRKPGSVVAAAAAEAKKKAVKESSIRSVQETVLPIKKRKTRE
TVSIEVKEVVKPLLVSTLGEKSGKGLKTCKSPGRKSKESSPKGRSSSASSPPKKEHHHHHHH
SESPKAPVPLLPPLPPPPPEPESSEDPTSPPEPQDLSSSVCKEEKMPRGGSLESDGCPKEPA
KTQPAVATAATAAEKYKHRGEGERKDIVSSSMPRPNREEPVDSRTPVTERVSS

Without wishing to be bound by theory, it is believed that the most significant functional regions of the MeCP2 protein are the methyl-CpG binding domain (MBD; underlined), the nuclear localisation signal (NLS; bold italics), and the NCoR/SMRT Interaction Domain (NID; double underlined). Thus, a MeCP2 protein will typically comprise:
(i) a methyl-CpG binding domain (MBD) having the sequence (SEQ ID NO: 5)

PAVPEASASPKQRRSIIRDRGPMYDDPTLPEGWTRKLKQRKSGRSAGKYD
VYLINPQGKAFRSKVELIAYFEKVGDTSLDPNDFDFTVTGRGSPSRREQK
PP

or a variant thereof having at least 70% identity, e.g. at least 75%, 80%, 85%, 90%, 95%, 96%, 97%, 98% or 99% identity thereto;
(ii) a NCoR/SMRT Interaction Domain (NID) having the sequence (SEQ ID NO: 6)

PGSVVAAAAAEAKKAVKESSIRSVQETVLPIKKRKTRETV

or a variant thereof having at least 70% identity, e.g. at least 75%, 80%, 85%, 90%, 95%, 96%, 97%, 98% or 99% identity thereto; and
(iii) a nuclear localisation signal (NLS).

Typically, the MBD is able to bind to methylated DNA. It is believed to possess a number of phosphorylation sites which shown in bold font and underlined above. These sites are Ser80, Ser86, Thr148, Ser149 and Ser164, numbered according to their positions in the human isoform 2 sequence.

The NID is typically able to interact with or bind to the NCoR/SMRT co-repressor complex.

The MBD is typically located N-terminal of the NID.

The NLS may be located between the MBD and NID.

The NLS may be the native MeCP2 NLS, having the sequence (SEQ ID NO: 7) RIKAEADPQAIPEKRGRK. However, many different NLS sequences are known and NLS sequences apart from the native MeCP2 NLS may be used, such as the SV40 Large T antigen NLS (SEQ ID NO: 8)(PKKKRKV).

Thus the MeCP2 protein may comprise or consist of the sequence (SEQ ID NO: 9):

SEDQDLQGLKDKPLKFKKVKKDKKEEKEGKHEPVQPSAHHSAEPAEAGKA
ETSEGSGSAPAVPEASASPKQRRSIIRDRGPMYDDPTLPEGWTRKLKQRK
SGRSAGKYDVYLINPQGKAFRSKVELIAYFEKVGDTSLDPNDFDFTVTGR
GSPSRREQKPPKKPKSPKAPGTGRGRGRPKGSGTTRPKAATSEGVQVKRV
LEKSPGKLLVKMPFQTSPGGKAEGGGATTSTQVMVIKRPGRKRKAEADPQ
AIPKKRGRKPGSVVAAAAAEAKKKAVKESSIRSVQETVLPIKKRKTRETV
SIEVKEVVKPLLVSTLGEKSGKGLKTCKSPGRKSKESSPKGRSSSASSPP
KKEHHHHHHKSESPKAPVPLLPPLPPPPPEPESSEDPTSPPEPQDLSSSV
CKEEKMPRGGSLESDGCPKEPAKTQPAVATAATAAEKYKHRGEGERKDIV
SSSMPRPNREEPVDSRTPVTERVSS;

or a functional variant thereof having at least 70% identity, e.g. at least 75%, 80%, 85%, 90%, 95%, 96%, 97%, 98% or 99% identity thereto; or a functional fragment of either. Differences from this sequence preferably lie outside the MBD and NID, and a functional NLS should be retained.

A fragment of this sequence, designated ΔNC, which is believed to be functionally equivalent has the sequence (SEQ ID NO: 10):

PAVPEASASPKQRRSIIRDRGPMYDDPTLPEGWTRKLKQRKSGRSAGKYD
VYLINPQGKAFRSKVELIAYFEKVGDTSLDPNDFDFTVTGRFSPSRREQK
PPKKPKSPKAPGTGRGRGRPKGSGTTRPKAATSEGVQVKRVLEKSPGKLL
VKMPFQTSPGGKAEGGGATTSTQVMIKRPGRRKRKAEADPQAIPKKRGRK
PGSVVAAAAAEAKKKAVKESSIRSVQETVLPIKKRKTRETV

Thus the MeCP2 protein may comprise or consist of the ΔNC sequence or may be a functional variant thereof having at least 70% identity, e.g. at least 75%, 80% 85%, 90%, 95%, 96%, 97%, 98% or 99% identity thereto. Again, differences from this sequence preferably lie outside the MBD and NID, and a functional NLS should be retained.

A further variant, designated ΔNIC, comprising the MBD and NID, and having an alternative NLS sequence (from SV40 large T antigen), but with much of the remaining native MeCP2 sequence deleted, has the sequence (SEQ ID NO: 11):

PAVPEASASPKQRRSIIRDRGPMYDDPTLPEGWTRKLKQRKSGRSAGKYD
VYLINPQGKAFRSKVELIAYFEKVGDTSLDPNDFDFTVTGRGSPSRREQK
PPGSSGSSGPKKKRKVPGSVVAAAAAEAKKKAVKESSIRSVQETVLPIKK
RKTRETV

Thus the MeCP2 protein may comprise or consist of the ΔNIC sequence or may be a functional variant thereof having at least 70% identity, e.g. at least 75%, 80%, 85%, 90%, 95%, 96%, 97%, 98% or 99% identity thereto. Again, differences from this sequence preferably lie outside the MBD and NID, and a functional NLS should be retained.

Where the MBD or NID of the MeCP2 protein contain one or more differences from the reference sequences provided, it may be desirable that those differences are conservative substitutions. It may also be desirable that the phosphorylation sites of the MBD are maintained.

The protein may further comprise an N-terminal portion having the sequence: (SEQ ID NO: 12) MALAALLAPSGGGGGGEEERLEEN or MVAGMLGEREEN (SEQ ID NO: 13), or at least 70%% identity to either, e.g. at least 75%, 80%, 85%, 90%, 95%, 96%, 97%, 98% or 99% identity, to either.

Thus, the MeCP2 protein encoded by the expression cassette may, for example, comprise or consist of one of the sequences:

(SEQ ID NO: 14)
MAAAAAAAPSGGGGGGEEERLEEKSEDQDLQGLKDKPKLFKKVKKDKKEE
KEGKHEPVQPSAHHSAEPAEAGKAETSEGSGSAPAVPEASASPKQRRSII
RDRGPMYDDPTLPEGWTRKLKQRKSGRSAGKYDVYLINPQGKAFRSKVEL
IAYFEKVGDTSLDPNDFDFTVTGRGSPSRREQKPPKKPKSPKAPGTGRGR
GRPKGSGTTRPKAATSEGVQVKRVLEKSPGKLLVKMPFQTSPGGKAEGGG
ATTSTQVMVIKRPGRKRKAEADPQAIPKKRGRKPGSVVAAAAAEAKKKAV
KESSIRSVQETVLPIKKRKTRETVSIEVKEVVKPLLVSTLGEKSGKGLKT
CKSPGRKSKESSPKGRSSSASSPPKKEHHHHHHHSESPKAPVPLLPPLPP
PPPEPESSEDPTSPPEPQDLSSSVCKEEKMPRGGSLESDGCPKEPAKTQP
AVATAATAAEKYKHRGEGERKDIVSSSMPRPNREEPVDSRTPVTERVSS
(human isoform 1)
(SEQ ID NO: 15)
MVAGMLGLREEKSEDQDLQGLKDKPLKFKKVKKDKKEEKEGKHEPVQPSA
HHSAEPAEAGKAETSEGSGSAPAVPEASASPKQRRSIIRDRGPMYDDPTL
PEGWTRKLKQRKSGRSAGKYDVYLINPQGKAFRSKVELIAYFEKVGDTSL
DPNDFDFTVTGRGSPSRREQKPPKKPKSPKAPGTGRGRGRPKGSGTTRPK
AATSEGVQVKRVLEKSPGKLLVKMPFQTSPGGKAEGGGATISTQVMVIER
PGRKRKAEADPQAIPKERGRKPGSVVAAAAAEAKKKAVKESSIRSVQETV
LPIKKRKTRETVSIEVKEVVKPLLVSTLGEKSGKGLKTCKSPGRKSKESS
PKGRSSSASSPEKKEHHHHHHHSESPKAPVPLLPPLPPPPPEPESSEDPT
SPPEPQDLSSSVCKEEEMPRGGSLESDGCPKEPAKTQPAVATAATAAEKY
KHRGEGERKDIVSSSMPRENREEPVDSRTPVTERVSS
(human isoform 2)
(SEQ ID NO: 16)
MAAAAAAAPSGGGGGGEEERLEEKPAVPEASASPKQRRSIIRDRGPMYDD
PTLPEGWTRKLKQRKSGRSAGKYDVYLINPQGKAFRSKVELIAYFEKVGD
TSLDENDFDFTVTGRGSPSRREQKPPKKPKSPKAPGTGRGRGRPKGSGTT
RPKAATSEGVQVKRVLEKSPGKLLVKMPFQTSPGGKAEGGGATTSTQVMV
IKRPGRKRKAEADPQAIPKKRGRKPGSVVAAAAAEAKKKAVKESSIRSVQ
ETVLPIKKRKTRETV
(ΔNC isoform 1)
(SEO ID NO: 17)
MVAGMLGLREEKPAVPEASASPKQRRSIIRDRGPMYDDPTLPEGWTRKLK
QRKSGRSAGKYDVYLINPQGKLFRSKVELIAYFEKVGDTSLDPNDFDFTV
TGRGSPSRREQKPPKKPKSPKAPGTGRGRGRPKGSGTTRPKAATSEGVQV
KRVLEKSPGKLLVKMPFQTSPGGKAEGGGATTSTQVMVIKRPGRKRKAEA
DPQAIPKKRGRKPGSVVAAAAAEAKKKAVKESSIRSVQETVLPIKKRKTR
ETV
(ΔNC isoform 2)
(SEQ ID NO: 18)
MAAAAAAAPSGGGGGGEEERLEEKPAVPEASASPKQRRSIIRDRGPMYDD
PILPEGWIRKLKQRKSGRSAGKYDVYLINPQGKAFRSKVELIAYFEKVGD
TSLDPNDFDFTVTGRGSPSRREQKPPGSSGSSGPKKKRKVPGSVVAAAAA
EAKKKAVKESSIRSVQETVLPIKKRKTRETV
(ΔNIC isoform 1)
(SEQ ID NO: 19)
MVAGMLGLREEKFAVPEASASPKQRRSIIRDRGPMYDDPILPEGWIRKLK
QRKSGRSAGKYDVYLINPQGKAFRSKVELIAYFEKVGDTSLDPNDFDFTV
TGRGSPSRREQKPPGSSGSSGPKKKRKVPGSVVAAAAAEAKKKAVKESSI
RSVQETVLPIKKRKTRETV
(ΔNIC isoform 2)

or may have at least 70% identity, e.g. at least 75%, 80%, 85%, 90%, 95%, 96%, 97%, 98% or 99% identity, to any one of those sequences.

Identity values of at least 80% or at least 90% to the reference sequences provided may be particularly preferred.

It will be apparent to the skilled person that the expression cassette of the invention may also usefully encode MeCP2 proteins from other species, especially other mammalian species, e.g. from a non-human primate, or a domestic, laboratory or livestock animal, such as a rodent (e.g. mouse, rat, guinea pig), lagomorph (e.g. rabbit), cat, dog, pig, cow, horse, sheep, goat, etc.

The MeCP2 protein may additionally comprise heterologous (i.e. non-MeCP2) sequence, e.g. at the C-terminal end of the molecule, such as an epitope tag to aid isolation or identification. Examples include a poly-histidine (e.g. hexa-histidine) tag, FLAG tag, Myc tag, fluorescent proteins such as green fluorescent protein (GFP) and enhanced green fluorescent protein (eGFP), etc. Such heterologous portions are typically no more than 50 amino acids in length, e.g. no more than 20 amino acids in length. For example, the MeCP2 protein encoded by the second generation vector described in the examples below comprises a C-terminal c-Myc epitope tag having the sequence EQKLISEEDL (SEQ ID NO: 20). This protein has the sequence (SEQ ID NO: 21):

MAAAAAAAPSGGGGGGEEERLEEKSEDQDLQGLKDKPLKFKKVKKDKKE
EKEGKHEPVQPSAHHSAEPAEAGKAETSEGSGSAPAVPEASASPKQRRS
IIRDRGPMYDDPTLPEGWTRKLKQRKSGRSAGKYDVYLINPQGKAFRSK
VELIAYFEKVGDTSLDPNDFDFTVTGRGSPSRREQKPPKKPKSPKAPGT
GRGRGRPKGSGTTRPKAATSEGVQVKRVLEKSPGKLLVKMPFQTSPGGK
AEGGGATTSTQVMVIKRPGRKRKAEADPQAIPKKRGRKPGSVVAAAAAE
AKKKAVKESSIRSVQETVLPIKKRKTRETVSIEVKEVVKPLLVSTLGEK
SGKGLKTCKSPGRKSKESSPKGRSSSASSPPKKEHHHHHHHSESPKAPV
PLLPPLPPPPPEPESSEDPTSPPEPQDLSSSVCKEEKMPRGGSLESDGC
PKEPAKTQPAVATAATAAEKYKHRGEGERKDIVSSSMPRPNREEPVDSR
TPVTERVSSRGPFEQKLISEEDLVD

where the MeCP2 sequence is underlined and the c-Myc epitope tag is double-underlined.

A c-Myc-tagged version of the ΔNC protein described above may have the sequence (SEQ ID NO: 22):

MAAAAAAAPSGGGGGGEEERLEEKPAVPEASASPKQRRSIIRDRGPMYDD
PTLPEGWTRKLKQRKSGRSAGKYDVYLINPQGKAFRSKVELIAYFEKVGD
TSLDPNDFDFTVTGRGSPSRREQKPPKKPKSPKAPGTGRGRGRPKGSGTT
RPKAATSEGVQVKRVLEKSPGKLLVKMPFQTSPGGKAEGGGATTSTQVMV
IKRPGRKRKAEADPQAIPKKRGRKPGSVVAAAAAEAKKKAVKESSIRSVQ
ETVLPIKKRKTRETVGSSGSSGEQLKISEEDLVD

where the MeCP2 sequence is underlined and the c-Myc epitope tag is double-underlined.

A c-Myc-tagged version of the ΔNIC protein described above may have the sequence (SEQ ID NO: 23):

MAAAAAAAPSGGGGGGEEERLEEKPAVPEASASPKQRRSIIRDRGPMYDD
PTLPEGWTRKLKQRKSGRSAGKYDVYLINPQGKAFRSKVELIAYFEKVGD
TSLDPNDFDFTVTGRGSPSRREQKPPGSSGSSGPKKKRKVPGSVVAAAAA
EAKKKAVKESSIRSVQETVLPIKKRKTRETVGSSGSSGEQKLISEEDLVD

where the MeCP2 sequence is underlined and the c-Myc epitope tag is double-underlined.

It will be understood that a protein intended for therapeutic use will generally not contain such heterologous elements, to reduce immunogenicity and risk of other side-effects.

In some embodiments, the MeCP2 protein encoded by the expression cassette is not more than 600 amino acids in length, e.g. not more than 550, 540, 530 or 520 amino acids in length. Thus the open reading frame encoding the MeCP2 protein will be not more than 1803 bases in length (including the top codon), e.g. not more than 1653, 1623, 1593 or 1563 bases in length.

Percent (%) amino acid sequence identity between a candidate sequence and the reference sequences presented above is defined as the percentage of amino acid residues in the candidate sequence that are identical with the amino acid residues in the reference sequence, after aligning the sequences and introducing gaps, if necessary, to achieve the optimum alignment, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

A conservative substitution may be defined as a substitution within an amino acid class and/or a substitution that scores positive in the BLOSUM62 matrix.

According to one classification, the amino acid classes are acidic, basic, uncharged polar and nonpolar, wherein acidic amino acids are Asp and Glu; basic amino acids are Arg, Lys and His; uncharged polar amino acids are Asn, Gln, Ser, Thr and Tyr; and non-polar amino acids are Ala, Gly, Val, Leu, Ile, Pro, Phe, Met, Trp and Cys.

According to another classification, the amino acid classes are small hydrophilic, acid/acid amide/hydrophilic, basic, small hydrophobic and aromatic, wherein small hydrophilic amino acids are Ser, Thr, Pro, Ala and Gly; acid/acidamide/hydrophilic amino acids are Asn, Asp, Glu and Gln; basic amino acids are His, Arg and Lys; small hydrophobic amino acids are Met, Ile, Leu and Val; and aromatic amino acids are Phe, Tyr and Trp

Substitutions which score positive in the BLOSUM62 matrix are as follows:

| | Original Residue | | | | | | | | | | | | | | | | | | | |
|---|---|---|---|---|---|---|---|---|---|---|---|---|---|---|---|---|---|---|---|---|
| | C | S | T | P | A | G | N | D | E | Q | H | R | K | M | I | L | V | F | Y | W |
| Substitution | — | T | S | — | S | — | S | N | D | E | N | Q | E | I | M | M | M | Y | H | F |
| | | A | | | | | D | E | Q | R | Y | K | Q | L | L | I | I | W | F | Y |
| | | N | | | | | H | | K | K | | | R | V | V | V | L | | W | |

### 5′ Transcriptional Control Region

The MeCP2 expression cassette comprises a 5′ transcriptional control region capable of directing transcription in neural cells, e.g. in neurons, especially of the brain and CNS. It may be desirable that expression also takes place in glial cells, although high levels of expression in glial cells are generally not preferred.

The 5′ transcriptional control region comprises a promoter and a transcriptional initiation site. It may also contain other control elements, including enhancer and/or silencer elements.

It may be possible to use a universal promoter such as a viral promoter (e.g. the SV40 promoter) or a mammalian “housekeeping” promoter. Preferably, though, the promoter directs expression preferentially in neural cells as compared to other cell types.

The second generation expression cassette described in the examples employs a contiguous stretch of chromosomal sequence from the human MeCP2 gene including a core promoter region, a silencer region upstream of (and slightly overlapping) the core promoter region) and a CNS regulatory element located downstream of the core promote region and upstream of the transcriptional start site. These sequences are illustrated in FIG. 14.

Without wishing to be bound by theory, it is believed that the core promoter and CNS regulatory element ensure adequate expression in neural cells, while the silencer may prevent over-expression and hence toxicity, in both neural cells and in other cell types including glial cells and liver cells.

Thus the 5′ transcriptional control region may comprise a core amino acid sequence having the sequence (SEQ ID NO: 24):

AAACCAGCCCCTCTGTGCCCTAGCCGCCTCTTTTTTCCAAGTGACAGTAG
AACTCCACCAATCCGCAGCTGAATGGGGTCCGCCTCTTTTCCCTGCCTAA
ACAGACAGGAACTCCTGCCAATTGAGGGCG

or a functional fragment thereof, or a variant thereof having no more than 20 nucleotide changes, e.g. no more than 10 or no more than 5 nucleotide changes compared to that sequence, wherein the core amino acid sequence region is capable of initiating transcription in neural cells, e.g. in neurons in the brain and/or CNS, alone or in conjunction with a CNS regulatory element

The 5′ transcriptional control region may comprise a silencer element having the sequence (SEQ ID NO: 25):

TTAAGCGCCAGAGTCCACAAGGGCCCAGTTAATCCTCAACATTCAAATGC
TGCCCACAAAAC

or a variant thereof having no more than 10 or no more than 5 nucleotide changes compared to that sequence.

The 5′ transcriptional control region may comprise a CNS regulatory element having the sequence (SEQ ID NO: 26):

CAGCACACAGGCTGGTCGG.

An E-box element (sequence CAGGTG) overlaps the 3′ end of the core promoter sequence, and an SP1 site (GGGCGG) is located 3′ of the CNS regulatory element. These elements may also be present if desired.

The 5′ transcriptional control region may comprise the native MeCP2 core promoter sequence annotated as mMeP426 in FIG. 14.

### MicroRNA Binding Sites

The 3′UTR of the MeCP2 expression cassette may contain one or more microRNA (miRNA) binding sites, to facilitate regulation of MeCP2 transgene expression.

MicroRNAs are small non-coding RNAs that have a substantial impact on cellular function through repression of translation (either through inhibition of translation or induction of mRNA degradation). MicroRNAs derive from primary RNA transcripts (pri-miRNA) synthesised by RNA pol II, which may be several thousand nucleotides in length. A single pri-miRNA transcript may give rise to more than one active miRNA.

In the nucleus, the Type III RNAse enzyme Drosha processes the pri-miRNA transcript into a precursor miRNA (pre-miRNA) consisting of a stem-loop or hairpin structure, normally around 70 to 100 nucleotides in length. The pre-miRNA is then transported to the cytoplasm, where it is processed further by the RNAse Dicer, removing the loop and yielding a mature double stranded miRNA molecule, having an active “guide” strand (typically 15 to 25 nucleotides in length) hybridised to a wholly or partially complementary “passenger” strand.

The mature double stranded miRNA is then incorporated into the RNA-induced silencing complex, where the guide strand hybridises to a binding site in the target mRNA.

The guide strand may not be completely complementary to the target binding site. However, a region of the guide strand designated the “seed” sequence is usually fully complementary to the corresponding “core” sequence of the target binding site. The seed sequence is typically 2 to 8 nucleotides in length and located at or near (within 1 or two nucleotides of) the 5′ end of the guide strand.

Without wishing to be bound by any particular theory, a miR-22 binding site may regulate MeCP2 expression in peripheral cells. See ref. 32.

A miR-22 binding site typically comprises at least the core sequence:

GGCAGCT.

For example, a miR-22 binding site may comprise the sequence (SEQ ID NO: 27):

ACAAGAATAAAGGCAGCTGTTGTCTCTTC

in which the core sequence is underlined;
or may differ therefrom at one or more positions, e.g. at up to 5 positions, up to 10 positions, up to 15 positions or up to 20 positions, all of which must lie outside the core sequence.

A miR-19 binding site may regulate MeCP2 expression in glial cells. See ref. 33.

A miR-19 binding site typically comprises at least the core sequence:

TTTGCAC.

For example, a miR-19 binding site may comprise the sequence (SEQ ID NO: 28):

AGAAGTAGCTTTGCACTTTTCTAAACTAGG

in which the core sequence is underlined;
or may differ therefrom at one or more positions, e.g. at up to 5 positions, up to 10 positions, up to 15 positions or up to 20 positions, all of which must lie outside the core sequence.

A miR-124 binding site may also regulate MeCP2 transgene expression in glial cells. See refs. 35 and 44.

A miR-124 binding site typically comprises at least the core sequence:

TGCCTTA.

A miR-124 binding site may comprise further sequences 5′ or 3′ of the core sequence if desired, but the core sequence will typically not vary.

A miR-132 binding site may regulate MeCP2 expression via a feedback loop with BNDF2 (brain-derived neurotrophic factor 2). MeCP2 is believed to increase expression levels of BNDF2. BNDF2 in turn increases levels of miR132, which is a negative regulator of MeCP2 expression. See ref. 34.

A miR132 binding site typically comprises at least the core sequence:

GACTGTTA.

For example, a miR-132 binding site may comprise the sequence (SEQ ID NO: 29):

AATATCACCAGGACTGTTACTCAATGTGTG

in which the core sequence is underlined;
or may differ therefrom at one or more positions, e.g. at up to 5 positions, up to 10 positions, up to 15 positions or up to 20 positions, all of which must lie outside the core sequence.

### AU-Rich Element

The 3′UTR of the expression cassette may encode an AU-rich element.

AU-rich elements (AREs) are common regulators of mRNA stability, via the 3′-5′ exosome pathway, and are typically located in the 3′UTR.

An AU-rich element may contain one or more repeats of the sequence AUUUA. It may also contain one or more so-called US2B elements, having the sequence AUAUAU.

The 3′UTR of the MeCP2 gene contains an AU-rich element having the sequence AUAUAUUUAAAAA (SEQ ID NO: 30)(ref. 38) and containing one AUUUA repeat and one ES2B element. Variations to this sequence may be possible.

### Other Regulatory Signals

The skilled person will be capable of designing other elements of the expression cassette to achieve appropriate expression of the MeCP2 transgene in the desired target cell type.

For example, transcriptional termination and polyadenylation signals will typically be present, to direct cleavage of the primary transcript and polyadenylation of the resulting mRNA. These may comprise “upstream elements” (5′ of the cleavage site) and “downstream elements” (3′ of the cleavage site).

A common upstream element is typically a hexamer located 10 to 30 nucleotides upstream of the cleavage site, and often referred to simply as a polyadenylation or poly(A) signal. The MeCP2 gene has two polyadenylation signals of which the sequence of the distal polyadenylation signal (UAUAAA) may be preferred. The sequence AAUAAA is also a commonly used polyadenylation signal.

A downstream element may be a U-rich or GU-rich element. These may contain binding sites for components of the polyadenylation machinery such as CstF (cleavage stimulation factor). The MeCP2 gene contains a GU-rich region having the sequence (SEQ ID NO: 31) UGUCCGUUUGUGUCUUUUGUUGU and containing two CstF binding sites each having the sequence UUUGU.

It will be understood that U (uridine) is referred to in the context of RNA sequences. Corresponding DNA sequences, e.g. as found in an AAV vector genome, will incorporate T instead.

The expression cassette will typically also contain a translational initiation signal, e.g. a Kozak sequence. The Kozak sequence includes the initiation codon for the MeCP2 protein, typically AUG. An example of a suitable Kozak sequence, used in the vectors described in the Examples below, is AAACCAUGG, where the initiation codon is underlined.

The native Kozak sequence from the human MECP2 gene has the sequence AAAAUGG, i.e. it lacks the CC doublet present in the vectors described below. The CC doublet was introduced to provide better conformity with the generally recognised consensus Kozak sequence and hence increase the strength of the Kozak sequence. However, given that high levels of MeCP2 expression can be deleterious, at least in some tissues, it may be desirable in some instances to use the native Kozak sequence rather than the modified sequence.

The skilled person will be aware that considerable variation within the Kozak sequence is possible and will be able to select further alternative sequences as appropriate.

### Pharmaceutical Compositions and Routes of Administration

The nucleic acids, virions, etc. described herein can be formulated in pharmaceutical compositions.

Administration may be peripheral, e.g. intravenous, cutaneous or subcutaneous, nasal, intramuscular or intraperitoneal, or direct to the central nervous system (CNS), e.g. by intrathecal injection or intra-cranial injection.

Intravenous and intrathecal administration may be preferred.

Pharmaceutical compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration.

For intravenous, cutaneous or subcutaneous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Compositions for direct administration to the CNS are typically minimal compositions lacking preservatives and other excipients, and may be specially prepared at the time of administration.

Administration is preferably in a “prophylactically effective amount” or a “therapeutically effective amount” (as the case may be), this being sufficient to show benefit to the individual. The actual amount administered, and rate and time-course of administration, may depend on the individual subject and the nature and severity of their condition.

Prescription of treatment, e.g. decisions on dosage etc., is within the responsibility of medical practitioners and other medical doctors, and typically takes account of the disorder to be treated, the condition of the individual patient, the site of delivery, the method of administration and other factors known to practitioners.

Examples of the techniques and protocols mentioned above can be found in Remington's Pharmaceutical Sciences, 20th Edition, 2000, pub. Lippincott, Williams & Wilkins.

### EXAMPLES

Dose Escalation with AAV/MECP2 Revealed a Narrow Therapeutic Window Following Systemic Administration.

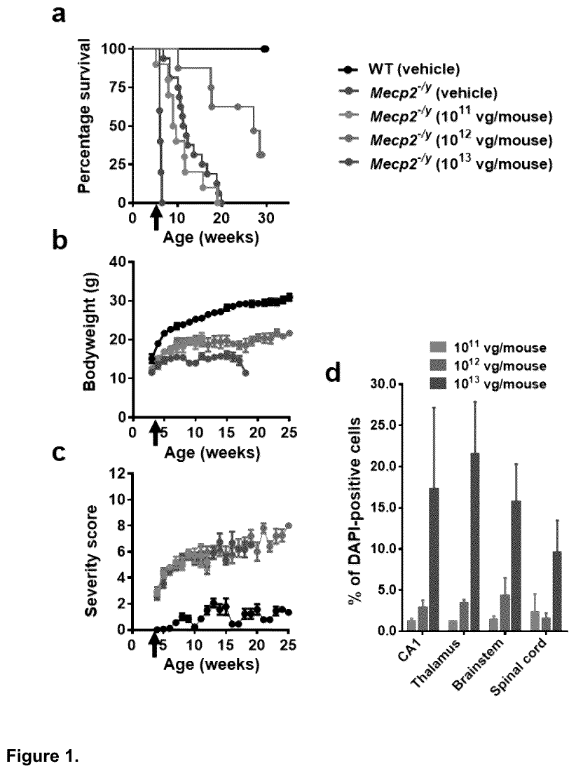

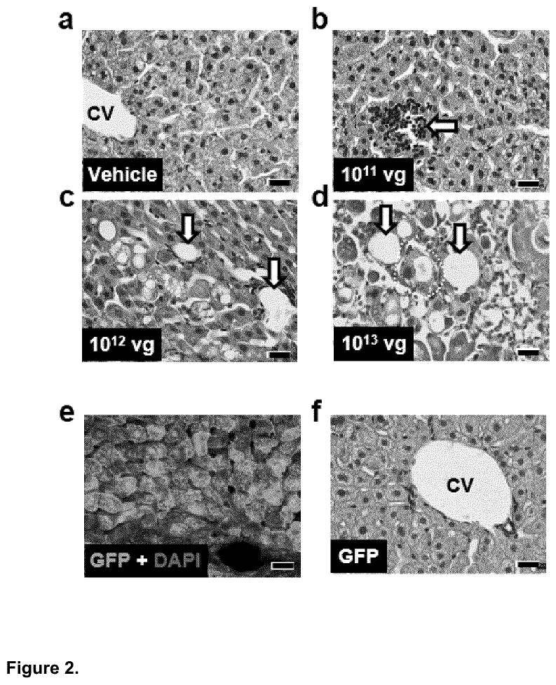

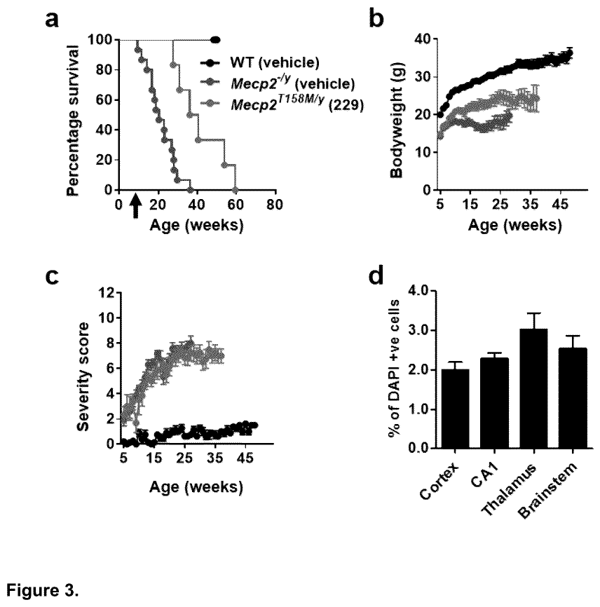

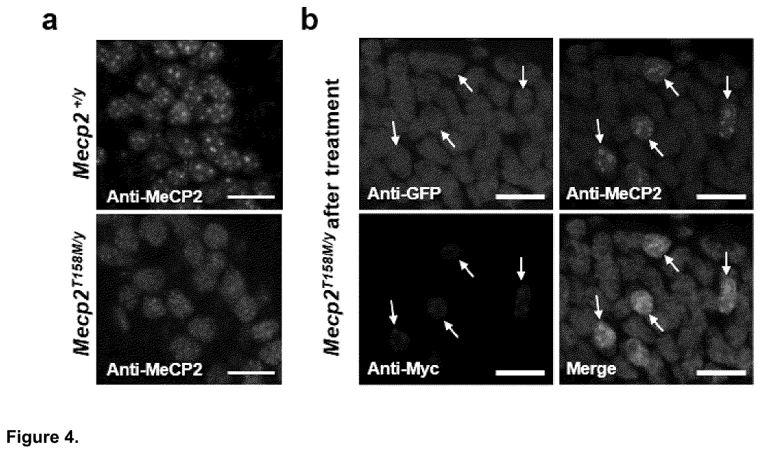

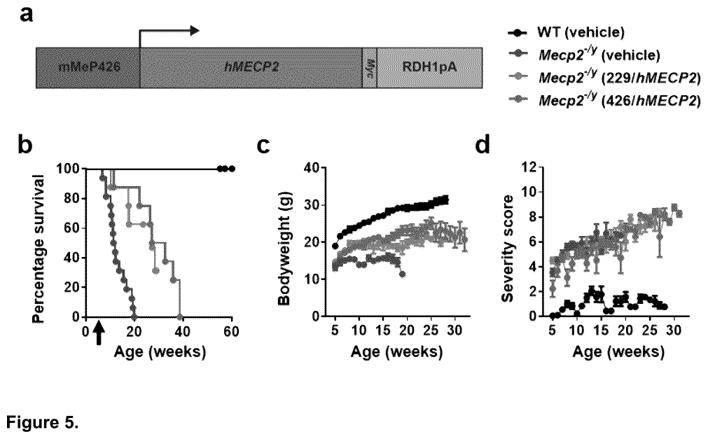

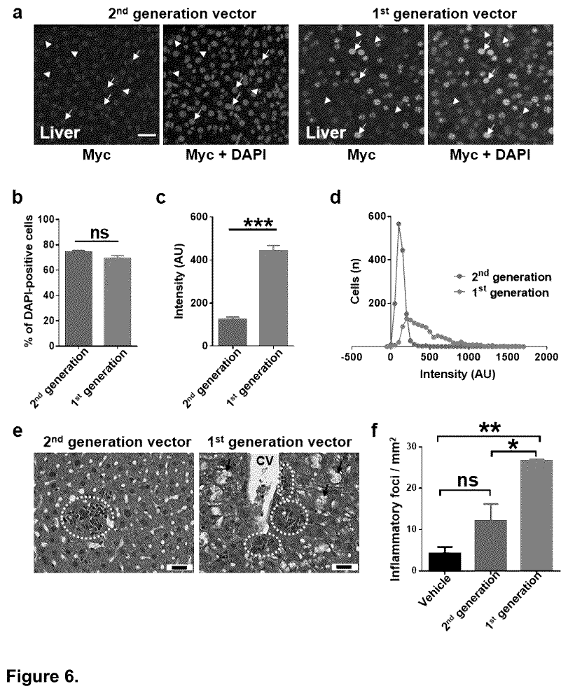

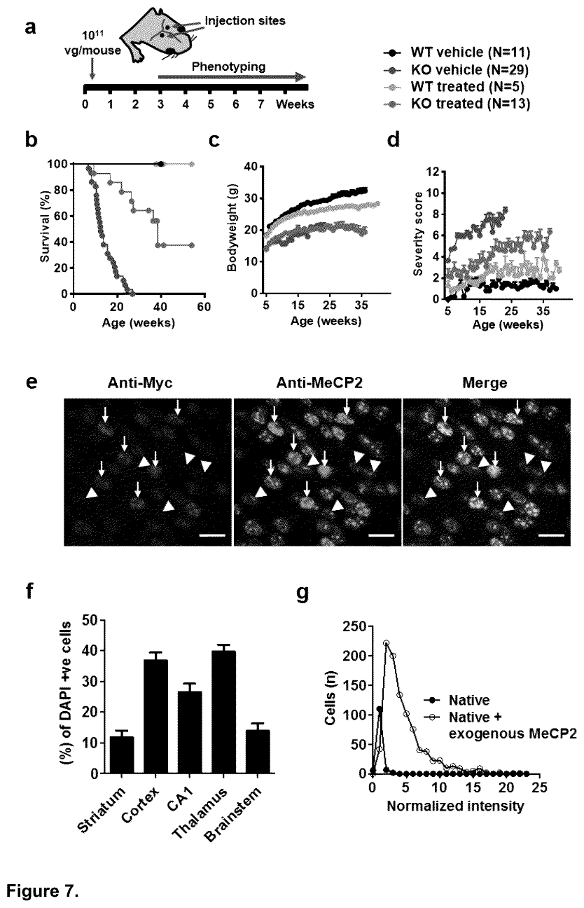

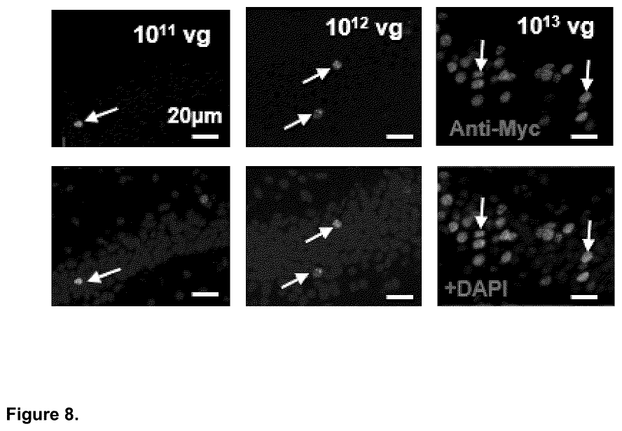

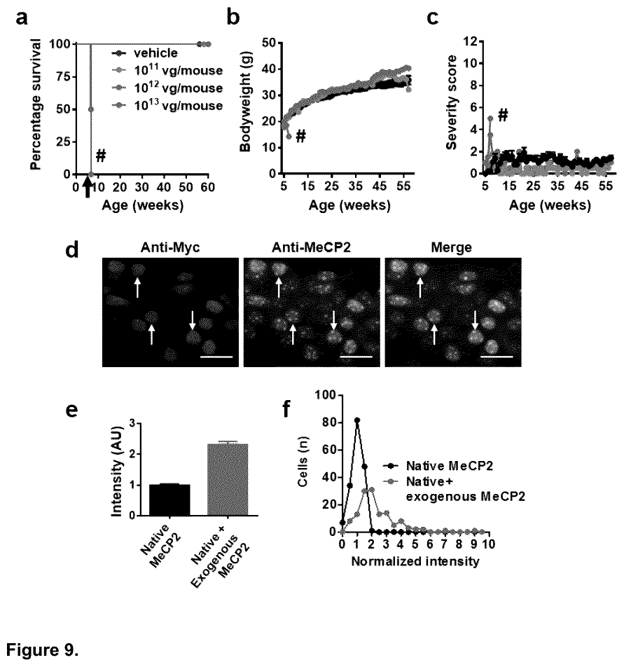

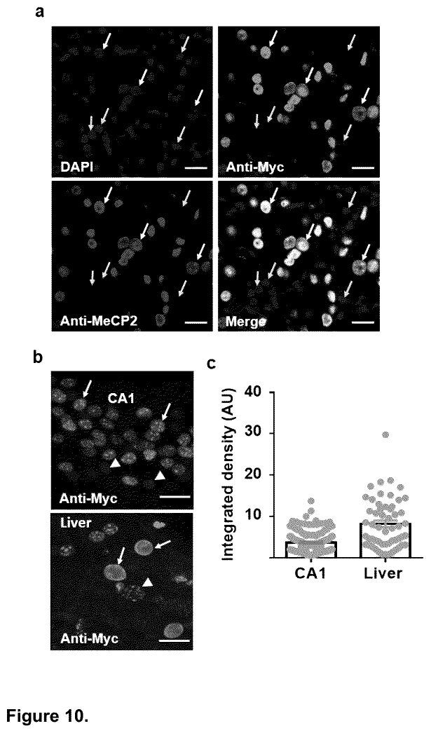

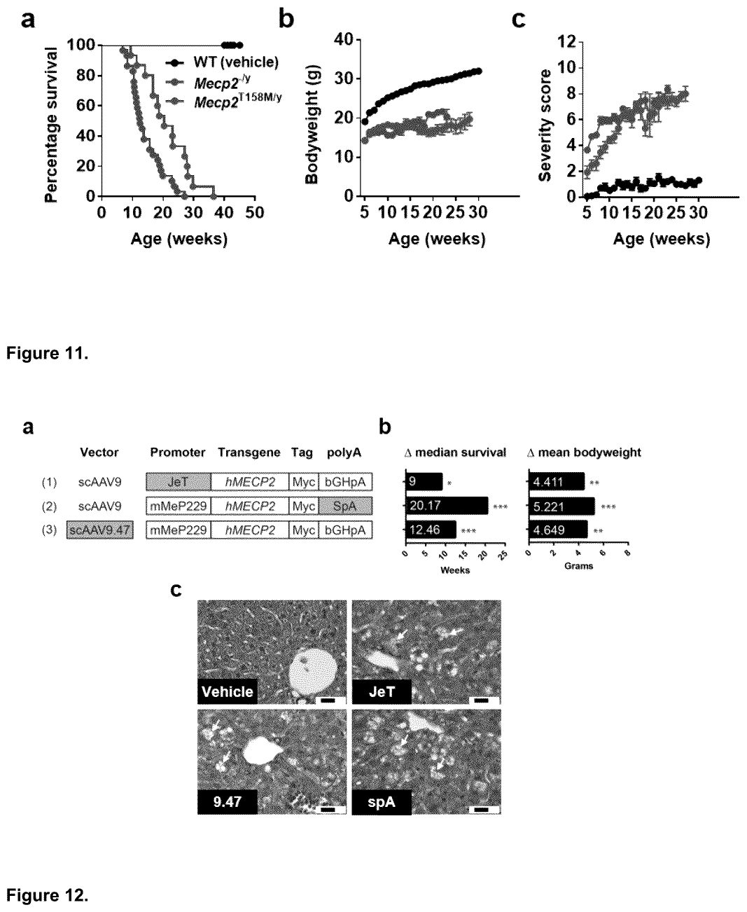

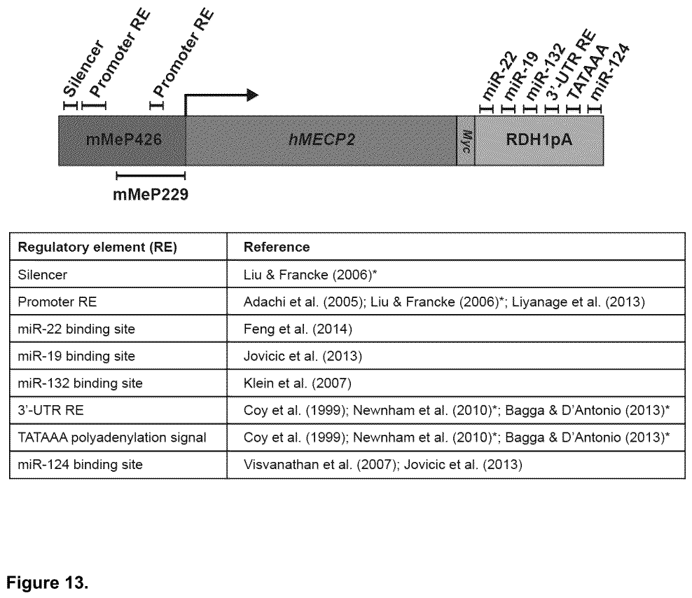

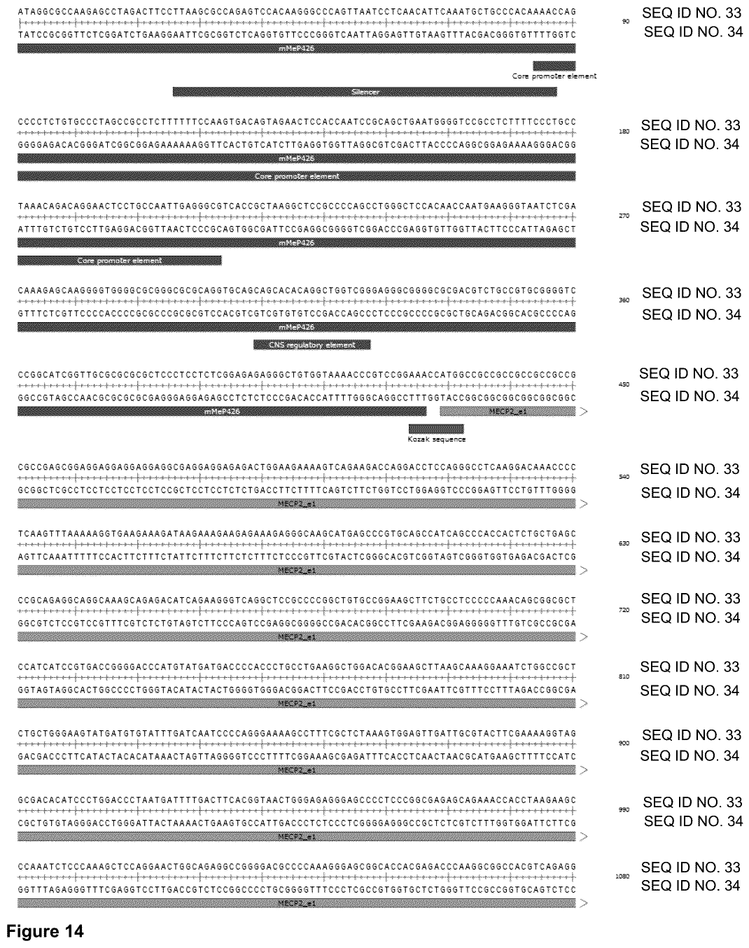

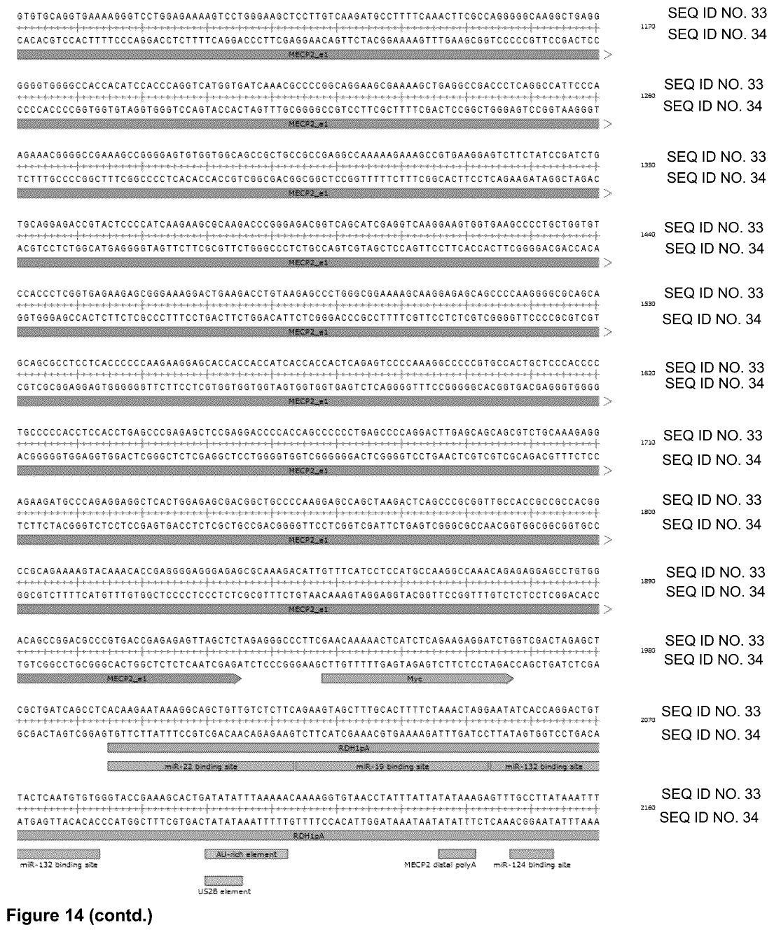

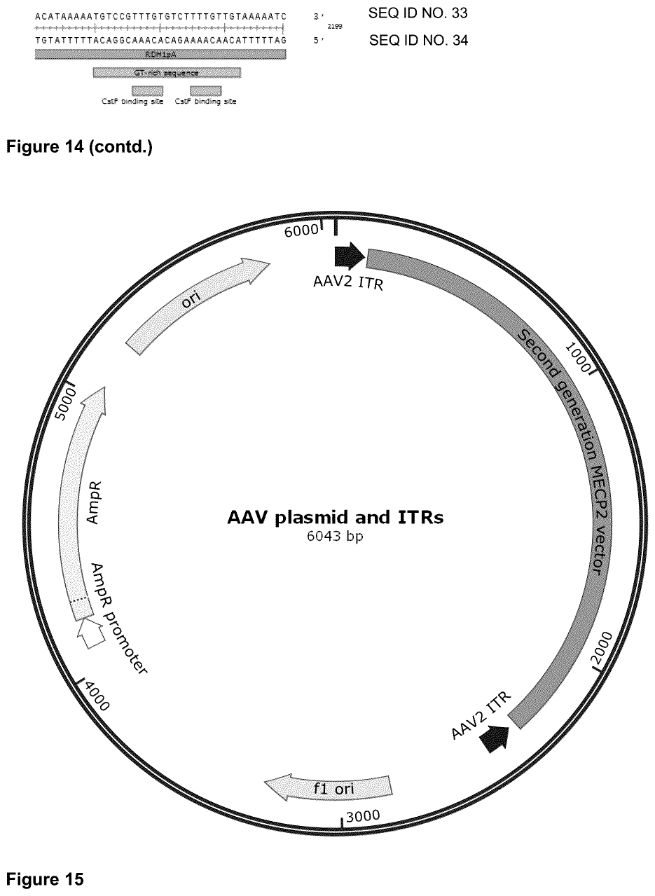

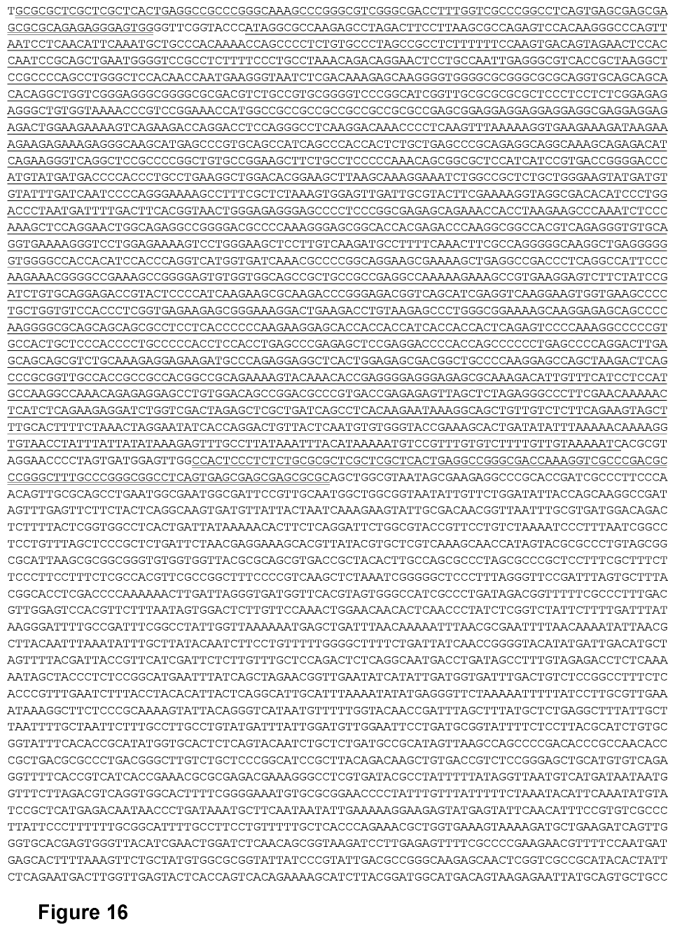

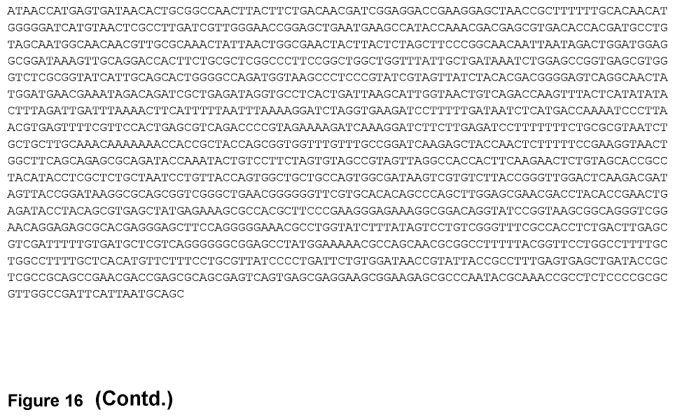

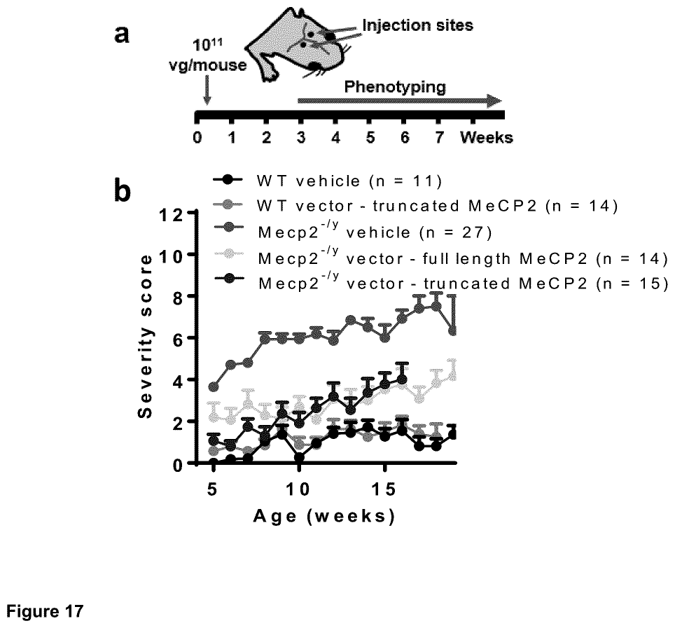

In order to explore the relationship between vector dose and therapeutic benefits, we conducted a dose escalation experiment in which an scAAV2/9 vector was used to deliver a Myc-tagged human MECP2_e1 cDNA under the control of a short, 229 bp region of the murine Mecp2 endogenous core promoter (MeP229)^{19, 22} henceforth referred to as the ‘1^{st} generation vector’. Juvenile male Mecp2^{-/y} and wild-type (WT) mice were injected at the age of 4-5 weeks into the tail vein either with vehicle or with 1×10¹¹ (low dose), 1×10¹² (moderate dose) or 1×10¹³ (high dose) viral genomes (vg) per mouse (dose range ˜1×10¹³-1×10¹⁵ vg/kg). As expected from previous studies of this knockout line 6,7,15, onset of RTT-like phenotypic signs in vehicle control-treated 15 Mecp2^{-/y} mice was observed from 4-weeks of age and severity progressively increased until death or censoring of all mice by 20 weeks of age (FIG. 1a-c). Mecp2^{-/y} mice treated with the low dose were undistinguishable from vehicle-injected mice in terms of survival, bodyweight and severity score (FIG. 1a-c). In contrast, Mecp2^{-/y} mice treated with the moderate dose (1×10¹²) showed significantly increased survival and bodyweight compared to the vehicle controls (median survival=27.3 weeks vs 11.64 weeks, p=0.001, Mantel-Cox test, FIG. 1a; p<0.05 for mean bodyweight measured at 11 weeks of age, the median survival for the control Mecp2^{-/y} mice, FIG. 1b). However, there was no difference in the RTT-like phenotype severity score at this dose (FIG. 1c). Finally, the cohort receiving the highest dose showed acute toxicity and lethality at 10-15 days post-injection (FIG. 1a).

Patterns of transduction in treated Mecp2^{-/y} mice were assessed within the CNS by anti-Myc antibody immunofluorescence labeling (FIG. 8), which revealed exogenous MeCP2 protein expression distributed in a punctate pattern within cell nuclei corresponding to that observed for endogenous MeCP2 in WT mice. Samples from the low dose cohort revealed low transduction efficiencies across brain regions (0.5 to 1%). The moderate dose resulted in ˜3-5% transduction efficiency, whereas the efficiency for the high dose was 10-22% (FIG. 1d).

In order to measure cellular levels of exogenous MeCP2 relative to native levels, WT mice were treated with vector as above. The low and moderate doses were tolerated and had no observable effect on bodyweight or phenotypic severity score (FIG. 9a-c). However, WT mice treated with the high dose exhibited the acute toxicity and rapid lethality observed in the knockout mice (FIG. 9a-c). Quantification of cellular levels of MeCP2 in mice given this high dose revealed that transduced hippocampal pyramidal cells expressed exogenous MeCP2 at a mean level equivalent to 120% of the endogenous level, resulting in total cellular levels of MeCP2 just over 2-fold higher than normal for these cells (FIG. 9d-f).

### Systemic Delivery of 1^{st} Generation Vector Resulted in Liver Toxicity.

To further investigate toxic effects encountered after systemic injection of the 1^{st} generation vector at high doses, levels of exogenous MeCP2 expression were tested in a range of peripheral tissues. Immunohistochemistry revealed that the proportion of Myc-positive cells in the liver was high (FIG. 10). Endogenous MeCP2 levels are known to be much lower in liver cells than in brain neurons^{23, 24} and are typically below detection threshold for immunohistochemistry using available antibodies (FIG. 10a). However, exogenous MeCP2 levels in a subset of liver cells (using anti-Myc-immunolabelling) of treated WT mice were found to be higher than MeCP2 levels seen in neurons (FIG. 10b-c) and were thus ˜20 times higher than levels found endogenously in such cells. Myc-positive cells were detected also in the heart, kidney and other peripheral tissues in treated Mecp2^{-/y} mice (data not shown).

Histological investigation of liver sections from mice injected with vehicle or low dose of the vector showed largely normal liver structure with occasional areas of mononuclear infiltration (FIG. 2a-b). In contrast, mice injected with higher doses of the vector showed a dose-dependent increase in pathological features including cellular destruction and vacuolation, loss of hepatocytes and mononuclear cell infiltration (FIG. 2c-d).

To address whether the observed liver pathology was due to the high copy number of viral particles per se or was a consequence of MeCP2 overexpression, we injected mice with a vector driving expression of GFP, but otherwise identical to the 1^{st} generation vector. Despite detection of widespread GFP expression in the liver (FIG. 2e), histological examination of liver sections revealed no evidence of cellular damage or immune cell infiltration (FIG. 2f). In addition, no changes in RTT aggregate severity score were observed with this vector (data not shown).

### Systemic Administration of 1^{st} Generation Vector Improves Survival in Mecp2[T158M] Knock-In Mice.

An important question for gene transfer in RTT is whether the presence of endogenous mutant MeCP2 might reduce the therapeutic effect of exogenous MeCP2. Male mice expressing native MeCP2 tagged with GFP as a fusion protein and harboring the common RTT-causing p.T158M mutation, Mecp2^{T158M/y 9}, display a phenotype very similar to that of Mecp2-null mice (FIG. 11) but with somewhat enhanced survival (median survival of 20.3 weeks and 12.4 weeks, respectively; p=0.0016, Mantel-Cox test).

Intravenous delivery of a moderate dose (1×10¹² vg/mouse) of the 1^{st} generation vector to 4-5 week old Mecp2^{T158M/y} mice resulted in significantly increased survival (FIG. 3a; median survival=38.3 weeks in vector-treated mice vs 20.3 weeks in vehicle-treated mice; p=0.0019, Mantel-Cox test, n=8-15 per group). There was a modest increase in bodyweight in the vector-treated cohort (FIG. 3b; p<0.05, one-way ANOVA using data at 20 weeks of age). However, there was no difference in RTT-like aggregate severity score between groups (FIG. 3c), consistent with a low brain transduction efficiency (˜2-4%) as revealed by anti-Myc labelling (FIG. 3d).

The p.T158M mutation affects the chromatin binding capacity of MeCP2, leading to loss of the punctate element of MeCP2 labelling in the nucleus (FIG. 4a)⁹. Immunolabelling of hippocampal neurons from treated Mecp2^{T158M/y} mice showed WT patterns of MeCP2 expression, with restored localization to DAPI bright spots, only in transduced (Myc-positive) cells (FIG. 4b). This is consistent with exogenous MeCP2 being able to localize normally to heterochomatin, despite the presence of mutant endogenous MeCP2 protein within the same nucleus.

Development of a 2^{nd} Generation Vector that Reduced Liver Toxicity after Systemic Administration

In light of the data described above, it was evident that a higher AAV vector dose is required to achieve therapeutically relevant levels of brain transduction after systemic delivery. However, severe toxicity after delivery of high doses of our 1^{st} generation cassette necessitated a new design. We tested a range of modifications to the expression cassette and capsid that were predicted to result in lower cellular expression levels and/or reduce liver tropism. This included the use of expression cassettes utilizing (1) an alternative, compact, and presumably weaker, JeT promoter²⁵, (2) a short synthetic polyadenylation (SpA) signal²⁶, FIG. 12a), and (3) the original 1^{st} generation expression cassette packaged in a scAAV9.47 capsid, which emerged from an in vivo screen for liver de-targeted capsid sequences relative to AAV9^{27, 28}. Systemic injection of these vectors at the moderate dose (1×10¹² vg/mouse) into 4-5 week old Mecp2^{-/y} mice resulted in significantly extended survival and improved bodyweight but there was no impact on the RTT-like aggregate severity score (FIG. 12b). In summary, none of these modifications resulted in any significant improvements over the 1^{st} generation vector (p>0.05 for all measures; ANOVA and Mantel Cox tests). Importantly, these modified vectors all caused the development of liver pathology similar to that observed with the first generation vector (as previously shown in FIG. 2; FIG. 12c).

The rationale for using an endogenous Mecp2 core promoter fragment (MeP229) in the 1^{st} generation vector was that it had been shown largely to recapitulate the endogenous tissue-level pattern of MeCP2 expression²². However, this core promoter fragment is missing a number of predicted upstream regulatory elements that may be important in cell-type specific regulation of MeCP2 expression²⁹⁻³¹. Therefore, we designed a 2^{nd} generation vector (v2) in which we used an extended promoter fragment (MeP426) incorporating additional promoter regulatory elements and a putative silencer element (FIG. 13). We predicted that this might better enable the regulation of exogenous MeCP2 levels in transduced cells. In addition to the extended promoter, we also incorporated a novel 3′-UTR consisting of a fragment of the endogenous MECP2 3′UTR together with a selected panel of binding sites for miRNAs known to be involved in regulation of Mecp2³²⁻³⁵ (FIG. 13).

In order to test the therapeutic efficacy of the 2^{nd} generation vector, a moderate dose (1×10¹² vg/mouse) was injected intravenously into 4-5 week old Mecp2^{-/y} mice. There was a significant extension of survival in the vector-treated compared to the vehicle-treated mice (median survival=29.9 weeks and 11.6 weeks, respectively; p<0.0001, Mantel-Cox, FIG. 5b). There was also significant improvement in bodyweight at the age of 11 weeks (p<0.05, one-way ANOVA, with Tukey's post-hoc pairwise comparison test, FIG. 5c). In contrast, there was no effect on RTT-like aggregate severity score (FIG. 5d). The 2^{nd} generation vector thus showed no therapeutic advantages over the 1^{st} generation vector after systemic delivery (FIG. 5b-d).

In order to compare this vector head-to-head with the 1^{st} generation vector in terms of liver safety, mice were injected intravenously with either 1^{st} or 2^{nd} generation vector at a dose of 1×10¹² vg/mouse. These mice were sacrificed after 30 days and tissues analysed for exogenous MeCP2 expression (using anti-Myc tag antibody) and signs of liver pathology (FIG. 6). There was no significant difference in transduction efficiency between vector constructs (FIG. 6b), but cellular levels of exogenous MeCP2 (anti-Myc) in mice treated with 1^{st} generation vector were significantly higher than those in mice treated with 2^{nd} generation vector (FIG. 6c; p<0.001, unpaired t-test). Analysis of the distribution of cellular MeCP2 expression levels in transduced cells showed that MeCP2 expression was more tightly regulated in mice injected with the 2^{nd} generation vector (FIG. 6d), with fewer cells exhibiting very high expression levels. Moreover, there was none of the disrupted hepatic architecture or vacuolation previously observed with the 1^{st} generation vector (FIG. 6e). The density of inflammatory foci was significantly higher in liver samples from mice injected with 1^{st} generation vector than those injected with the 2^{nd} generation vector (FIG. 6f).

### Neonatal Cerebroventricular Injection of the 2n d Generation Vector Improved RTT-Like Aggregate Severity Score

The lack of impact on the phenotype after systemic administration is consistent with the low brain transduction efficiencies observed, as it has been established that phenotype severity and degree of improvement after gene restoration correlate with the proportion of MeCP2-expressing cells in the brain¹⁶. We therefore decided to test the 2^{nd} generation vector by direct cerebroventricular injection in mouse neonates, a delivery route that is known to afford widespread transgene expression¹⁹. When delivered at a dose of 1×10¹¹ vg/mouse (FIG. 7a), there was a pronounced extension in the survival of Mecp2^{-/y} mice treated with the 2^{nd} generation vector in comparison to vehicle-treated mice (median survival=38.5 and 12.4 weeks, respectively; p<0.0001, Mantel-Cox test, FIG. 7b). Whilst there was a negligible effect of vector on bodyweight (FIG. 7c), an important observation was the clear improvement in the RTT-like aggregate severity score compared to vehicle-treated Mecp2-null mice (FIG. 7d). Exogenous MeCP2 (revealed by anti-Myc tag immunolabelling) was detectable in all brain regions, with transduction efficiencies across brain regions ranging from ˜10-40% (FIG. 7e-f). Distribution analysis revealed that the modal cellular MeCP2 level in transduced cells in cortex was approx. twice that of endogenous MeCP2 (consistent with an exogenous expression level equal to the endogenous level), with some cells expressing higher levels of exogenous MeCP2 (FIG. 7g). The ΔNIC protein described above was shown to yield comparable results to the full length MeCP2 protein (FIG. 17).

### Discussion

The reversal of a wide range of RTT-like phenotypes in mice following delayed unsilencing of Mecp2 provides a strong rationale for gene transfer as a therapeutic strategy in RTT^{15, 16}. There are likely to be a variety of barriers to translational success that will need to be identified and addressed in order to secure optimal outcomes in human clinical trials. In the current study, we identified particular challenges associated with systemic delivery of a MECP2-bearing gene therapy vector in terms of a narrow therapeutic window driven by low brain transduction efficiency and the appearance of peripheral overexpression toxicity upon further dose escalation. However, peripheral overexpression can be reduced by refining the cassette design. We show that direct brain delivery of vector in neonatal mice can achieve therapeutically relevant levels of transduction that result in phenotype amelioration. We also show that the vector has similar effectiveness in mice expressing the most common RTT-causing mutation suggesting that the presence of existing mutant forms of MeCP2 is unlikely to be an obstacle to translational success. These results are consistent with experiments in transgenic mice expressing both mutant and WT forms of the protein³⁶.

Recent attempts to deliver MECP2 exogenously in mouse models of RTT used widely varying vector doses but are difficult to compare based on additional differences in cassette design and other variables including viral production, dosing protocol and phenotype measures¹⁹⁻²¹ In the current study, we used our previously published cassette design (human MECP2_e1 under the control of a MeP229 core promoter fragment),¹⁹ to directly investigate the effect of dose in terms of efficacy and safety. A notable finding was the overall lack of efficacy across the range of doses tested in terms of an effect on RTT-like phenotype severity score. This is not due to such phenotypes being inherently resistant to reversal^{15, 16} but is instead most likely explained by the low levels of brain transgene expression afforded by this route of delivery. In contrast to the phenotype severity score, there was a clear dose-response relationship for survival, with the intermediate dose causing a modest increase in mean bodyweight and a significant extension in survival. It is not clear whether the survival and bodyweight effects are due to sufficient (if low) transduction levels in critical brain regions or to expression of MeCP2 in peripheral tissues relevant to mortality. Recent evidence suggests that MeCP2 levels in peripheral tissues can subtly affect bodyweight²³ and it is possible that this may indirectly affect survival measures, as we are obliged to use acute loss of bodyweight as an end-point criterion. Another potential explanation is that we were underestimating levels of transduction efficiency related to survival based on the sensitively of our immunohistochemical detection. However, vector biodistribution validation using qPCR was consistent with our measurements, confirming very modest transduction following systemic delivery. Only the highest dose tested produced appreciable levels of brain transduction (>10-20%), and, unfortunately, the severe liver pathology and lethality associated with this does precluded assessment of the potential for brain specific therapeutic effects in this situation. Liver cells normally express relatively low levels of MeCP2 compared to neurons²³ and identical doses of a GFP-expressing vector were not toxic, so the dose-dependent liver pathology is likely be attributed to the overexpression of exogenous MeCP2.

Our initial attempts to lower toxic MeCP2 expression and/or reduce liver tropism involved modifications to the expression cassette and capsid. However, the use of putative weaker synthetic promoters and polyadenylation signals were not sufficient to avoid liver toxicity. Surprisingly, the use of an AAV9.47 capsid, which is purported to de-target the liver relative to AAV9^{27, 28}, resulted in liver pathology similar to that seen with AAV9. We therefore focused efforts on a 2^{nd} generation vector, whose design was based on the inclusion of endogenous regulatory elements that may better regulate levels of exogenous MeCP2 in transduced cells. This included the incorporation of an extended endogenous promoter and an endogenous 3′-UTR fragment. Studies analyzing the well-conserved human MECP2 and mouse Mecp2 promoter regions indicated the presence of a number of putative regulatory elements within a 1 kb window immediately upstream of the transcription start site²⁹⁻³¹. Consequently, our extended endogenous promoter (426 bp) in the 2^{nd} generation vector comprised a putative silencer element at position −274 to −335, with respect to the RefSeq transcription start site (FIG. 13).

An endogenous 3′-UTR was also incorporated, containing the distal MECP2 polyadenylation signal and a number of clustered putative regulatory elements³⁷⁻³⁹. In addition, we performed an analysis of miRNA binding sites in the 3′-UTR of MECP2 using a number of bioinformatic tools⁴⁰⁻⁴² and incorporated a compact sequence containing binding sites of three highly conserved miRNAs known to be involved in regulation of MeCP2 in the brain; miR-22⁴³, miR-19⁴⁴ and miR-132³⁴. Combined, these modifications significantly reduced MeCP2 expression in the liver with subsequent reduction of the hepatotoxicity encountered with the 1^{st} generation vector. The relative importance of different modifications (elements within the extended promoter and novel 3′-UTR) were not investigated. However, the efficacy of both vectors after systemic injection of moderate doses was not significantly different. The important advantage of the 2^{nd} generation vector is the lack of prominent liver pathology at a dose that provides some therapeutic benefit (i.e. 1×10¹² vg/mouse). The improved survival after systemic injection, despite low brain transduction efficiency, could be due to restoration of MeCP2 levels in sufficiently numerous critical cells in the brain, or due to restoration in important peripheral tissues. Targeting more cells in the brain through direct brain injection in mouse neonates, along with potentially greater impact via earlier intervention, led to pronounced survival enhancement at a dose (1×10¹¹ vg/mouse) approximately equivalent to the 10¹² systemic dose. Delivery by this direct brain injection route was associated with an improvement in bodyweight but, importantly, also with an improvement in RTT-like phenotype score. The improvement was not as profound as that reported in genetic reversal experiments 16 and this is likely to be due to the combined effects of (1) the relative inefficiency of MeCP2 re-expression across the brain (10-40%) compared to genetic reversal experiments (up to 90%) and (2) the possible deleterious counteracting effects of overexpressing MeCP2 in a proportion of transduced cells. Analysis of MeCP2 levels indeed indicates a significant pool of cells overexpressing MeCP2, presumably transduced with multiple copies of vector delivering MECP2. This may also account for the slightly elevated severity score in vector-treated WT mice (FIG. 7d) in the form of mild hindlimb clasping. Overall, the proof-of-concept experiments involving direct brain delivery in neonatal mice suggest that if transduction efficiency across the brain can reach sufficiently high levels, then a behavioral improvement is conferred by this vector design.

### CONCLUSION

The results of the current study highlight the challenges associated with both systemic and direct brain delivery of MECP2. The findings suggest that achieving widespread brain expression, whilst at the same time maintaining cell-type appropriate control of MeCP2 levels, will be essential requirements for the successful development of a translational therapy. The development of expression cassettes capable of producing effective and sub-toxic levels of MeCP2 may overcome issues of cellular overexpression and enable direct delivery via the cerebrospinal fluid compartment. Whilst AAV9 appears to be insufficiently efficient in terms of brain transduction after systemic delivery of MECP2 to achieve the desired therapeutic benefit, combining the safer 2^{nd} generation cassette together with capsids with improved brain penetrance⁴⁵ may effectively pair effective CNS gene transfer with safe levels of peripheral MeCP2 transgene expression. Such a combination would hold enhanced translational promise.

### Material and Methods

### Animals

All experiments were carried out in accordance with the European Communities Council Directive (86/609/EEC) and with the terms of a project license under the UK Scientific Procedures Act (1986). The Mecp2-null, Mecp2^{tml.lBird} and Mecp2^{T158M} mice, originally provided as a kind gift from Professor Adrian Bird, were maintained on a C57BL/6 background. Animals were maintained on 12-hour light/dark cycles with free access to normal mouse food. Mice were genotyped as described previously^{9, 15}.

### Viral Vector Preparation.

Recombinant AAV vector particles were generated at the UNC Gene Therapy Center Vector Core facility. Self-complementary AAV (scAAV) particles (AAV2 ITR-flanked genomes packaged into AAV9 or AAV9.47 serotype capsids) were produced from suspension HEK293 cells transfected using polyethyleneimine (Polysciences, Warrington, PA) with helper plasmids (pXX6-80, pGSK2/9) and a plasmid containing the appropriate ITR-flanked transgene construct. All MeCP2-expressing constructs utilized the human MECP2_e1 coding region with a C-terminal Myc epitope tag unless stated otherwise. Virus production was performed as previously described⁴⁶, and the vectors were prepared in a final formulation of high-salt phosphate-buffered saline (PBS; containing 350 mmol/l total NaCl) supplemented with 5% sorbitol.

scAAV Vector Injection and Mouse Phenotyping.

Frozen scAAV9 viral particle aliquots were thawed and diluted to 100 μl in PBS/350 mmol/1 NaCl containing 5% sorbitol. Control injections were made using the same diluent lacking vector (‘vehicle control’). For direct brain injection into mouse neonates, littermates were sexed at birth and direct bilateral injections of virus (3 μl per site) were delivered into the neuropil of unanaesthetised P0-3 males, as described previously¹⁹. The injected pups were returned to the home cage containing their non-injected female littermates. Genotyping was carried out at 3 weeks, at which time phenotyping was initiated. For injection into juvenile male mice, injections were made via the tail vein at 4-5 weeks of age. Following injection, all mice were weighed weekly. Phenotyping was carried out, blind to genotype and treatment, twice a week. Mice were scored on an aggregate severity scale using an established protocol (mice were scored for RTT-like phenotypes, comprising mobility, gait, breathing, hindlimb clasping, tremor and general condition;^{15, 16, 19, 21} For survival analysis, mice were censored after natural death or if bodyweight losses exceeded 20% of peak bodyweight.

### Immunohistochemistry

Mice were anaesthetized with pentobarbitone (50 mg, intraperitoneally) and transcardially perfused with 4% paraformaldehyde (0.1 mol/l PBS). A vibrating microtome (Leica VT1200; Leica, Milton Keynes, UK) was used to obtain 80 μm sections of brain, spinal cord, and liver. Sections were washed three times in 0.3 mol/l PBS and were then transferred to 10 mM sodium citrate (pH 6, 85° C., 30 minutes) for antigen retrieval. Sections were then incubated in the blocking solution (5% normal goat serum in 0.3 mol/l PBS with 0.3% Triton X-100) for 1 hour at room temperature. Samples then were incubated for 48 hours on a shaker at 4° C. with the following primary antibodies: rabbit anti-Myc (Abcam, ab9106); mouse monoclonal anti-MeCP2 (Sigma, WH0004204M1), chicken anti GFP, Abcam ab13970). The primary antibodies were then washed off (3×0.3 mol/l PBST) and secondary antibodies applied to the sections overnight at 4° C.: Alexa fluor 488 goat anti-mouse/rabbit (Invitrogen, Carlsbad, CA; 1/500), Alexa fluor 546 goat anti-mouse/rabbit (Invitrogen; 1/500), Alexa fluor 649, Goat anti mouse (Jackson immunoresearch, 112-495-003JIR). Finally, sections were incubated with 4′,6-diamidino-2-phenylindole (DAPI) nuclear stain (Sigma, Poole, UK; 1/1,000) for 30 minutes at room temperature before mounting with Vectashield (Vector labs, Peterborough, UK).

### Hematoxylin and Eosin (H&E) Staining

Liver samples were rinsed with 0.1 mol/1 PBS then dehydrated through ascending grades of ethanol and cleared in amyl acetate using an automated tissue processor. Specimens were embedded in paraplast and sections (10 μm thick) were collected on APES (aminopropyltriethoxysilane) coated slides and dried overnight in the oven at 37° C. Sections were then deparaffinised through two changes of Histo-clear (Agar Scientific, UK) for 15 min and rehydrated through descending grades of alcohol (100%, 90%, and 70%). The sections were stained with Mayer's hematoxylin for 8 min and then rinsed using tap water. The nuclei were stained blue by placing the slides into Scott's solution for 1 min and then rinsed using tap water. Sections were then stained with 1% eosin for 2 min and washed by water. Finally, the sections were dehydrated through ascending grades of alcohol and histoclear before being mounted with DPX. Images were captured using an AxioCam MRc (Zeiss, Germany) mounted on a light microscope (Zeiss, Germany).

### Image Analysis

Analysis of expression patterns, transduction efficiency, and quantification of exogenously derived MeCP2 levels within nuclei was carried out on image stacks captured using a Zeiss LSM710 or Zeiss Axiovert LSM510 laser confocal microscope (Zeiss, Cambridge, UK). Z-series were taken at 1 μm intervals through the section of interest using a 40× objective. To estimate transduction efficiency, images were captured as above and the ratio of Myc-immunopositive nuclei to DAPI-stained nuclei was calculated for random fields (n=12 images/region: 4 images from each of three mice) from sections of hippocampus (CA1 region), layer 5 of primary motor cortex, thalamus, hypothalamus, brainstem, and striatum. To quantify levels of exogenously derived MeCP2 per nucleus in WT mice, confocal stacks (20 μm thick) were obtained as above and ImageJ software (http://rsbweb.nih.gov/ij/) was used to determine mean MeCP2-channel fluorescence intensity within transduced (Myc +ve) and non-transduced (Myc −ve) cells. Fluorescence in the DAPI channel was used to define the nuclear boundary.

### Statistical Analysis

Tests for differences between treatment groups were carried out in GraphPad PRISM using one-way ANOVA, Student's t-test, and Mantel-Cox test (survival curves), as appropriate. p<0.05 was used to define statistical significance. In multi group comparisons, multiple testing correction for pairwise tests amongst groups was applied using Tukey's post-hoc analysis.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

The teaching of all references in the present application, including patent applications and granted patents, are herein fully incorporated by reference. Any patent application to which this application claims priority is incorporated by reference herein in its entirety in the manner described herein for publications and references.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance. The term “about” (or “around”) in all numerical values allows for a 5% variation, i.e. a value of about 1.25% would mean from between 1.19%-1.31%.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Neul, J L, Kaufmann, W E, Glaze, D G, Christodoulou, J, Clarke, A J, Bahi-Buisson, N, et al. (2010). Rett syndrome: revised diagnostic criteria and nomenclature. Ann Neurol 68: 944-950.
2. Amir, R E, Van den Veyver, I B, Wan, M, Tran, C Q, Francke, U, and Zoghbi, H Y (1999). Rett syndrome is caused by mutations in X-linked MECP2, encoding methyl-CpG-binding protein 2. Nat Genet 23: 185-188.
3. Bienvenu, T, and Chelly, J (2006). Molecular genetics of Rett syndrome: when DNA methylation goes unrecognized. Nat Rev Genet 7: 415-426.
4. Lyst, M J, and Bird, A (2015). Rett syndrome: a complex disorder with simple roots. Nat Rev Genet 16: 261-275.
5. Chen, R Z, Akbarian, S, Tudor, M, and Jaenisch, R (2001). Deficiency of methyl-CpG binding protein-2 in CNS neurons results in a Rett-like phenotype in mice. Nat Genet 27: 327-331.
6. Guy, J, Hendrich, B, Holmes, M, Martin, J E, and Bird, A (2001). A mouse Mecp2-null mutation causes neurological symptoms that mimic Rett syndrome. Nat Genet 27: 322-326.
7. Shahbazian, M, Young, J, Yuva-Paylor, L, Spencer, C, Antalffy, B, Noebels, J, et al. (2002). Mice with truncated MeCP2 recapitulate many Rett syndrome features and display hyperacetylation of histone H3. Neuron 35: 243-254.
8. Goffin, D, Allen, M, Zhang, L, Amorim, M, Wang, I T, Reyes, A R, et al. (2011). Rett syndrome mutation MeCP2 T158A disrupts DNA binding, protein stability and ERP responses. Nat Neurosci 15: 274-283.
9. Brown, K, Selfridge, J, Lagger, S, Connelly, J, De Sousa, D, Kerr, A, et al. (2016). The molecular basis of variable phenotypic severity among common missense mutations causing Rett syndrome. Hum Mol Genet 25: 558-570.
10. Lyst, M J, Ekiert, R, Ebert, D H, Merusi, C, Nowak, J, Selfridge, J, et al. (2013). Rett syndrome mutations abolish the interaction of MeCP2 with the NCoR/SMRT co-repressor. Nat Neurosci 16: 898-902.
11. Pitcher, M R, Herrera, J A, Buffington, S A, Kochukov, M Y, Merritt, J K, Fisher, A R, et al. (2015). Rett syndrome like phenotypes in the R255X Mecp2 mutant mouse are rescued by MECP2 transgene. Hum Mol Genet 24: 2662-2672.
12. Archer, H, Evans, J, Leonard, H, Colvin, L, Ravine, D, Christodoulou, J, et al. (2007). Correlation between clinical severity in patients with Rett syndrome with a p.R168X or p.T158M MECP2 mutation, and the direction and degree of skewing of X-chromosome inactivation. J Med Genet 44: 148-152.
13. Ghosh, R P, Horowitz-Scherer, R A, Nikitina, I, Gierasch, L M, and Woodcock, C L (2008). Rett syndrome-causing mutations in human MeCP2 result in diverse structural changes that impact folding and DNA interactions. J Biol Chem 283: 20523-20534.
14. Leonard, H, Cobb, 5, and Downs, J (2016). Clinical and biological progress over 50 years in Rett syndrome. Nat Rev Neurol.
15. Guy, J, Gan, J, Selfridge, J, Cobb, S, and Bird, A (2007). Reversal of neurological defects in a mouse model of Rett syndrome. Science 315: 1143-1147.
16. Robinson, L, Guy, J, McKay, L, Brockett, E, Spike, R C, Selfridge, J, et al. (2012). Morphological and functional reversal of phenotypes in a mouse model of Rett syndrome. Brain 135: 2699-2710.
17. Jugloff, D G, Vandamme, K, Logan, R, Visanji, N P, Brotchie, J M, and Eubanks, J H (2008). Targeted delivery of an Mecp2 transgene to forebrain neurons improves the behavior of female Mecp2-deficient mice. Hum Mol Genet 17: 1386-1396.
18. Gadalla, K K E, Ross, P D, Hector R. D., Bahey, N G, Bailey, M E S, and Cobb, S R (2015). Gene therapy for Rett syndrome: prospects and challenges. Future Neurology 10: 467-484.
19. Gadalla, K K, Bailey, M E, Spike, R C, Ross, P D, Woodard, K T, Kalburgi, S N, et al. (2013). Improved survival and reduced phenotypic severity following AAV9/MECP2 gene transfer to neonatal and juvenile male Mecp2 knockout mice. Mol Ther 21: 18-30.
20. Matagne, V, Ehinger, Y, Saidi, L, Borges-Correia, A, Barkats, M, Bartoli, M, et al. (2016). A codon-optimized Mecp2 transgene corrects breathing deficits and improves survival in a mouse model of Rett syndrome. Neurobiol Dis 99: 1-11.
21. Garg, S K, Lioy, D T, Cheval, H, McGann, J C, Bissonnette, J M, Murtha, M J, et al. (2013). Systemic delivery of MeCP2 rescues behavioral and cellular deficits in female mouse models of Rett syndrome. J Neurosci 33: 13612-13620.
22. Gray, S J, Foti, S B, Schwartz, J W, Bachaboina, L, Taylor-Blake, B, Coleman, J, et al. (2011). Optimizing promoters for recombinant adeno-associated virus-mediated gene expression in the peripheral and central nervous system using self-complementary vectors. Hum Gene Ther 22: 1143-1153.
23. Ross, P D, Guy, J, Selfridge, J, Kamal, B, Bahey, N, Tanner, K E, et al. (2016). Exclusive expression of MeCP2 in the nervous system distinguishes between brain and peripheral Rett syndrome-like phenotypes. Hum Mol Genet.
24. Skene, P J, Illingworth, R S, Webb, S, Kerr, A R, James, K D, Turner, D J, et al. (2010). Neuronal MeCP2 is expressed at near histone-octamer levels and globally alters the chromatin state. Mol Cell 37: 457-468.
25. Tornoe, J, Kusk, P, Johansen, T E, and Jensen, P R (2002). Generation of a synthetic mammalian promoter library by modification of sequences spacing transcription factor binding sites. Gene 297: 21-32.
26. Levitt, N, Briggs, D, Gil, A, and Proudfoot, N J (1989). Definition of an efficient synthetic poly(A) site. Genes Dev 3: 1019-1025.
27. Pulicherla, N, Shen, S, Yadav, S, Debbink, K, Govindasamy, L, Agbandje-McKenna, M, et al. (2011). Engineering liver-detargeted AAV9 vectors for cardiac and musculoskeletal gene transfer. Mol Ther 19: 1070-1078.
28. Karumuthil-Melethil, S, Nagabhushan Kalburgi, S, Thompson, P, Tropak, M, Kaytor, M D, Keimel, J G, et al. (2016). Novel Vector Design and Hexosaminidase Variant Enabling Self-Complementary Adeno-Associated Virus for the Treatment of Tay-Sachs Disease. Hum Gene Ther 27: 509-521.
29. Liu, J, and Francke, U (2006). Identification of cis-regulatory elements for MECP2 expression. Human molecular genetics 15: 1769-1782.
30. Adachi, M, Keefer, E W, and Jones, F S (2005). A segment of the Mecp2 promoter is sufficient to drive expression in neurons. Human molecular genetics 14: 3709-3722.
31. Liyanage, V R, Zachariah, R M, and Rastegar, M (2013). Decitabine alters the expression of Mecp2 isoforms via dynamic DNA methylation at the Mecp2 regulatory elements in neural stem cells. Molecular autism 4: 46.
32. Feng, Y, Huang, W, Wani, M, Yu, X, and Ashraf, M (2014). Ischemic preconditioning potentiates the protective effect of stem cells through secretion of exosomes by targeting Mecp2 via miR-22. PLoS One 9: e88685.
33. Jovicic, A, Roshan, R, Moisoi, N, Pradervand, S, Moser, R, Pillai, B, et al. (2013). Comprehensive expression analyses of neural cell-type-specific miRNAs identify new determinants of the specification and maintenance of neuronal phenotypes. J Neurosci 33: 5127-5137.
34. Klein, M E, Lioy, D T, Ma, L, Impey, S, Mandel, G, and Goodman, R H (2007). Homeostatic regulation of MeCP2 expression by a CREB-induced microRNA. Nat Neurosci 10: 1513-1514.
35. Visvanathan, J, Lee, 5, Lee, B, Lee, J W, and Lee, S K (2007). The microRNA miR-124 antagonizes the anti-neural REST/SCP1 pathway during embryonic CNS development. Genes Dev 21: 744-749.
36. Heckman, L D, Chahrour, M H, and Zoghbi, H Y (2014). Rett-causing mutations reveal two domains critical for MeCP2 function and for toxicity in MECP2 duplication syndrome mice. Elife 3.
37. Coy, J F, Sedlacek, Z, Bachner, D, Delius, H, and Poustka, A (1999). A complex pattern of evolutionary conservation and alternative polyadenylation within the long 3″-untranslated region of the methyl-CpG-binding protein 2 gene (MeCP2) suggests a regulatory role in gene expression. Human molecular genetics 8: 1253-1262.
38. Bagga, J S, and D'Antonio, L A (2013). Role of conserved cis-regulatory elements in the post-transcriptional regulation of the human MECP2 gene involved in autism. Human genomics 7: 19.
39. Newnham, C M, Hall-Pogar, T, Liang, S, Wu, J, Tian, B, Hu, J, et al. (2010). Alternative polyadenylation of MeCP2: Influence of cis-acting elements and trans-acting factors. RNA biology 7: 361-372.
40. Lewis, B P, Burge, C B, and Bartel, D P (2005). Conserved seed pairing, often flanked by adenosines, indicates that thousands of human genes are microRNA targets. Cell 120: 15-20.
41. Vorozheykin, P S, and Titov, I I (2015). Web server for prediction of miRNAs and their precursors and binding sites. Mol Biol+49: 755-761.
42. Rehmsmeier, M, Steffen, P, Hochsmann, M, and Giegerich, R (2004). Fast and effective prediction of microRNA/target duplexes. Rna 10: 1507-1517.
43. Feng, Y L, Huang, W, Wani, M, Yu, X Y, and Ashraf, M (2014). Ischemic Preconditioning Potentiates the Protective Effect of Stem Cells through Secretion of Exosomes by Targeting Mecp2 via miR-22. PloS one 9.
44. Jovicic, A, Roshan, R, Moisoi, N, Pradervand, S, Moser, R, Pillai, B, et al. (2013). Comprehensive Expression Analyses of Neural Cell-Type-Specific miRNAs Identify New Determinants of the Specification and Maintenance of Neuronal Phenotypes. Journal of Neuroscience 33: 5127-5137.
45. Deverman, B E, Pravdo, P L, Simpson, B P, Kumar, S R, Chan, K Y, Banerjee, A, et al. (2016). Cre-dependent selection yields AAV variants for widespread gene transfer to the adult brain. Nature biotechnology 34: 204-209.
46. Clement, N, and Grieger, J C (2016). Manufacturing of recombinant adeno-associated viral vectors for clinical trials. Mol Ther Methods Clin Dev 3: 16002.
47. Gray, S J, Blake, B L, Criswell, H E, Nicolson, S C, Samulski, R J, and McCown, T J (2010). Directed Evolution of a Novel Adeno-associated Virus (AAV) Vector That Crosses the Seizure-compromised Blood-Brain Barrier (BBB). Mol Ther 18: 570-578.

1.-27. (canceled)

28. A method of treating Rett syndrome in a subject in need thereof, the method comprising administering to the subject an AAV vector comprising a MeCP2 expression cassette, the expression cassette comprising, in operable linkage from 5′ to 3′:
(a) a 5′ transcriptional control region comprising a promoter capable of driving transcription in neural cells, wherein the promoter is a MeCP2 promoter comprising a MeCP2 silencer element and a CNS regulatory element;
(b) a Kozak sequence;
(c) an open reading frame encoding a MeCP2 protein;
(d) a 3′ untranslated region (3′UTR) comprising a MeCP2 polyadenylation signal a binding site for miR-22, a binding site for miR-19, a binding site for miR-132, a binding site for miR-124, and one or more AU-rich element of the sequence AUUUA; and
(e) transcriptional termination signals; and
wherein the MeCP2 expression cassette is not more than about 5 kb in length.

29. The method of claim 28, wherein the encoded MeCP2 protein comprises the sequence:
(SEQ ID NO: 11)
PAVPEASASPKQRRSIIRDRGPMYDDPTLPEGWTRKLKQRKSGRSAGKYD
VYLINPQGKAFRSKVELIAYFEKVGDTSLDPNDFDFTVTGRGSPSRREQK
PPGSSGSSGPKKKRKVPGSVVAAAAAEAKKKAVKESSIRSVQETVLPIKK
RKTRETV.
or is a variant thereof having at least 70%, at least 75%, at least 80%, at least 85%, at least 90%, at least 95%, at least 96%, at least 97%, at least 98%, or at least 99% identity thereto.

30. The method of claim 28, wherein the MeCP2 protein further comprises an N-terminal portion having a sequence
(SEQ ID NO: 12)
MAAAAAAAPSGGGGGGEEERLEEK.

31. The method of claim 28, wherein the AAV vector is a self-complementary AAV vector.

32. The method of claim 28, wherein the AAV vector is an AAV9 vector.

33. The method of claim 28, wherein the AAV vector is administered intrathecally.

34. A method of enhancing expression of MeCP2 protein in a target cell comprising introducing into the target cell an AAV vector comprising a MeCP2 expression cassette, the expression cassette comprising, in operable linkage from 5′ to 3′:
(a) a 5′ transcriptional control region comprising a promoter capable of driving transcription in neural cells, wherein the promoter is a MeCP2 promoter comprising a MeCP2 silencer element and a CNS regulatory element;
(b) a Kozak sequence;
(c) an open reading frame encoding a MeCP2 protein;
(d) a 3′ untranslated region (3′UTR) comprising a MeCP2 polyadenylation signal a binding site for miR-22, a binding site for miR-19, a binding site for miR-132, a binding site for miR-124, and one or more AU-rich element of the sequence AUUUA; and
(e) transcriptional termination signals; and
wherein the MeCP2 expression cassette is not more than about 5 kb in length.

35. The method of claim 34, wherein the encoded MeCP2 protein comprises the sequence:
(SEQ ID NO: 11)
PAVPEASASPKQRRSIIRDRGPMYDDPTLPEGWTRKLKQRKSGRSAGKYD
VYLINPQGKAFRSKVELIAYFEKVGDTSLDPNDFDFTVTGRGSPSRREQK
PPGSSGSSGPKKKRKVPGSVVAAAAAEAKKKAVKESSIRSVQETVLPIKK
RKTRETV.
or is a variant thereof having at least 70%, at least 75%, at least 80%, at least 85%, at least 90%, at least 95%, at least 96%, at least 97%, at least 98%, or at least 99% identity thereto.

36. The method of claim 34, wherein the MeCP2 protein further comprises an N-terminal portion having a sequence
SEQ ID NO: 12)
MAAAAAAAPSGGGGGGEEERLEEK

37. The method of claim 34, wherein the AAV vector is a self-complementary AAV vector.

38. The method of claim 34, wherein the AAV vector is an AAV9 vector.

39. A method comprising:
testing a subject for the presence of one or more mutations in the MeCP2 gene indicative of the presence of, or a predisposition to, Rett syndrome; and,
if the subject has one or more mutations in the MeCP2 gene indicative of the presence of, or a predisposition to, Rett syndrome, administering to the subject an AAV vector comprising a MeCP2 expression cassette, the expression cassette comprising, in operable linkage from 5′ to 3′:
(a) a 5′ transcriptional control region comprising a promoter capable of driving transcription in neural cells, wherein the promoter is a MeCP2 promoter comprising a MeCP2 silencer element and a CNS regulatory element;
(b) a Kozak sequence;
(c) an open reading frame encoding a MeCP2 protein;
(d) a 3′ untranslated region (3′UTR) comprising a MeCP2 polyadenylation signal a binding site for miR-22, a binding site for miR-19, a binding site for miR-132, a binding site for miR-124, and one or more AU-rich element of the sequence AUUUA; and
(e) transcriptional termination signals; and
wherein the MeCP2 expression cassette is not more than about 5 kb in length.

40. The method of claim 39, wherein the encoded MeCP2 protein comprises the sequence:
(SEQ ID NO: 11)
PAVPEASASPKQRRSIIRDRGPMYDDPTLPEGWTRKLKQRKSGRSAGKYD
VYLINPQGKAFRSKVELIAYFEKVGDTSLDPNDFDFTVTGRGSPSRREQK
PPGSSGSSGPKKKRKVPGSVVAAAAAEAKKKAVKESSIRSVQETVLPIKK
RKTRETV.
or is a variant thereof having at least 70%, at least 75%, at least 80%, at least 85%, at least 90%, at least 95%, at least 96%, at least 97%, at least 98%, or at least 99% identity thereto.

41. The method of claim 39, wherein the MeCP2 protein further comprises an N-terminal portion having a sequence
(SEQ ID NO: 12)
MAAAAAAAPSGGGGGGEEERLEEK.

42. The method of claim 39, wherein the AAV vector is a self-complementary AAV vector.

43. The method of claim 39, wherein the AAV vector is an AAV9 vector.

44. The method of claim 39, wherein the AAV vector is administered intrathecally.

45. The method of claim 39, wherein the one or more mutations in the MeCP2 gene indicative of the presence of, or a predisposition to, Rett syndrome is a loss of function mutation.


SEQUENCE LISTING (34 sequences)

SEQ ID NO: 1 - DNA, 104 bp, synthetic construct
  misc_feature 1..104: note = scAAV vector containing AAV-2 ITR
  source 1..104: mol_type = other DNA
gcgcgctcgctcgctcactgaggccgcccgggcaaagcccgggcgtcgggcgacctttggtcgcccggcctcagtgagcgagcgagcgcgcagagagggagtgg

SEQ ID NO: 2 - DNA, 104 bp, synthetic construct
  misc_feature 1..104: note = scAAV vector containing AVV-2 ITRs
  source 1..104: mol_type = other DNA
ccactccctctctgcgcgctcgctcgctcactgaggccgggcgaccaaaggtcgcccgacgcccgggctttgcccgggcggcctcagtgagcgagcgagcgcgc

SEQ ID NO: 3 - protein, 499 aa, synthetic construct
  REGION 1..499: note = Human MeCP2 protein isoform 1
MAAAAAAAPSGGGGGGEEERLEEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSS

SEQ ID NO: 4 - protein, 487 aa, synthetic construct
  REGION 1..487: note = Human MeCP2 protein isoform 2
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSS

SEQ ID NO: 5 - protein, 102 aa, synthetic construct
  REGION 1..102: note = methyl-CpG binding domain (MBD) of human MeCP protein
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPP

SEQ ID NO: 6 - protein, 40 aa, synthetic construct
  REGION 1..40: note = NCoR/SMRT Interaction Domain (NID) of human MeCP2
PGSVVAAAAAEAKKAVKESSIRSVQETVLPIKKRKTRETV

SEQ ID NO: 7 - protein, 17 aa, synthetic construct
  REGION 1..17: note = native MeCP2 NLS
RKAEADPQAIPKKRGRK

SEQ ID NO: 8 - protein, 7 aa, synthetic construct
  REGION 1..7: note = SV40 Large T antigen NLS
PKKKRKV

SEQ ID NO: 9 - protein, 475 aa, synthetic construct
  REGION 1..475: note = MeCP2
SEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSS

SEQ ID NO: 10 - protein, 241 aa, synthetic construct
  REGION 1..241: note = MeCP2 fragment designated Delta NC
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETV

SEQ ID NO: 11 - protein, 157 aa, synthetic construct
  REGION 1..157: note = Protein variant designated Delta NIC
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETV

SEQ ID NO: 12 - protein, 24 aa, synthetic construct
  REGION 1..24: note = N-terminal portion
MAAAAAAAPSGGGGGGEEERLEEK

SEQ ID NO: 13 - protein, 12 aa, synthetic construct
  REGION 1..12: note = N-terminal portion
MVAGMLGLREEK

SEQ ID NO: 14 - protein, 499 aa, synthetic construct; identical to SEQ ID NO: 3
  REGION 1..499: note = MeCP2 protein encoded by the expression cassette, human isoform 1

SEQ ID NO: 15 - protein, 487 aa, synthetic construct; identical to SEQ ID NO: 4
  REGION 1..487: note = MeCP2 protein encoded by the expression cassette, human isoform 2

SEQ ID NO: 16 - protein, 265 aa, synthetic construct
  REGION 1..265: note = MeCP2 protein encoded by the expression cassette Delta NC isoform 1
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETV

SEQ ID NO: 17 - protein, 253 aa, synthetic construct
  REGION 1..253: note = MeCP2 protein encoded by the expression cassette Delta NC isoform 2
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETV

SEQ ID NO: 18 - protein, 181 aa, synthetic construct
  REGION 1..181: note = MeCP2 protein encoded by the expression cassette, Delta NIC isoform 1
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETV

SEQ ID NO: 19 - protein, 169 aa, synthetic construct
  REGION 1..169: note = MeCP2 protein encoded by the expression cassette, Delta NIC isoform 2
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETV

SEQ ID NO: 20 - protein, 10 aa, synthetic construct
  REGION 1..10: note = C-terminal c-Myc epitope tag
EQKLISEEDL

SEQ ID NO: 21 - protein, 515 aa, synthetic construct
  REGION 1..515: note = MeCP2 protein comprising a heterologous sequence
MAAAAAAAPSGGGGGGEEERLEEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSSRGPFEQKLISEEDLVD

SEQ ID NO: 22 - protein, 284 aa, synthetic construct
  REGION 1..284: note = A c-Myc-tagged version of the Delta NC protein
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVGSSGSSGEQKLISEEDLVD

SEQ ID NO: 23 - protein, 200 aa, synthetic construct
  REGION 1..200: note = c-Myc-tagged version of the Delta NIC protein
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETVGSSGSSGEQKLISEEDLVD

SEQ ID NO: 24 - protein, 130 aa, synthetic construct
  REGION 1..130: note = 5' transcriptional control comprising an amino acid sequence
AAACCAGCCCCTCTGTGCCCTAGCCGCCTCTTTTTTCCAAGTGACAGTAGAACTCCACCAATCCGCAGCTGAATGGGGTCCGCCTCTTTTCCCTGCCTAAACAGACAGGAACTCCTGCCAATTGAGGGCG

SEQ ID NO: 25 - DNA, 62 bp, synthetic construct
  misc_feature 1..62: note = 5' transcriptional control region comprising a silencer element
  source 1..62: mol_type = other DNA
ttaagcgccagagtccacaagggcccagttaatcctcaacattcaaatgctgcccacaaaac

SEQ ID NO: 26 - DNA, 19 bp, synthetic construct
  misc_feature 1..19: note = 5' transcriptional control region comprising a CNS regulatory element
  source 1..19: mol_type = other DNA
cagcacacaggctggtcgg

SEQ ID NO: 27 - DNA, 29 bp, synthetic construct
  misc_feature 1..29: note = a miR-22 binding site
  source 1..29: mol_type = other DNA
acaagaataaaggcagctgttgtctcttc

SEQ ID NO: 28 - DNA, 30 bp, synthetic construct
  misc_feature 1..30: note = a miR-19 binding site
  source 1..30: mol_type = other DNA
agaagtagctttgcacttttctaaactagg

SEQ ID NO: 29 - DNA, 30 bp, synthetic construct
  misc_feature 1..30: note = a miR-132 binding site
  source 1..30: mol_type = other DNA
aatatcaccaggactgttactcaatgtgtg

SEQ ID NO: 30 - RNA, 13 nt, synthetic construct
  misc_feature 1..13: note = 3'UTR of the MeCP2 gene, AU-rich element
  source 1..13: mol_type = other RNA
atatatttaaaaa

SEQ ID NO: 31 - RNA, 23 nt, synthetic construct
  misc_feature 1..23: note = MeCP2 gene, GU-rich region
  source 1..23: mol_type = other RNA
tgtccgtttgtgtcttttgttgt

SEQ ID NO: 32 - DNA, 6043 bp, synthetic construct
  misc_feature 1..6043: note = 2nd generation expression cassette
  source 1..6043: mol_type = other DNA
tgcgcgctcgctcgctcactgaggccgcccgggcaaagcccgggcgtcgggcgacctttggtcgcccggcctcagtgagcgagcgagcgcgcagagagggagtggggttcggtacccataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctctagagggcccttcgaacaaaaactcatctcagaagaggatctggtcgactagagctcgctgatcagcctcacaagaataaaggcagctgttgtctcttcagaagtagctttgcacttttctaaactaggaatatcaccaggactgttactcaatgtgtgggtaccgaaagcactgatatatttaaaaacaaaaggtgtaacctatttattatataaagagtttgccttataaatttacataaaaatgtccgtttgtgtcttttgttgtaaaaatcacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagc

SEQ ID NO: 33 - DNA, 2199 bp, synthetic construct
  misc_feature 1..2199: note = 2nd Generation Expression Cassette
  source 1..2199: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctctagagggcccttcgaacaaaaactcatctcagaagaggatctggtcgactagagctcgctgatcagcctcacaagaataaaggcagctgttgtctcttcagaagtagctttgcacttttctaaactaggaatatcaccaggactgttactcaatgtgtgggtaccgaaagcactgatatatttaaaaacaaaaggtgtaacctatttattatataaagagtttgccttataaatttacataaaaatgtccgtttgtgtcttttgttgtaaaaatc

SEQ ID NO: 34 - DNA, 2199 bp, synthetic construct
  misc_feature 1..2199: note = 2nd Generation Expression Cassette
  source 1..2199: mol_type = other DNA
tatccgcggttctcggatctgaaggaattcgcggtctcaggtgttcccgggtcaattaggagttgtaagtttacgacgggtgttttggtcggggagacacgggatcggcggagaaaaaaggttcactgtcatcttgaggtggttaggcgtcgacttaccccaggcggagaaaagggacggatttgtctgtccttgaggacggttaactcccgcagtggcgattccgaggcggggtcggacccgaggtgttggttacttcccattagagctgtttctcgttccccaccccgcgcccgcgcgtccacgtcgtcgtgtgtccgaccagccctcccgccccgcgctgcagacggcacgccccagggccgtagccaacgcgcgcgcgagggaggagagcctctctcccgacaccattttgggcaggcctttggtaccggcggcggcggcggcggcgcggctcgcctcctcctcctcctccgctcctcctctctgaccttcttttcagtcttctggtcctggaggtcccggagttcctgtttggggagttcaaatttttccacttctttctattctttcttctctttctcccgttcgtactcgggcacgtcggtagtcgggtggtgagacgactcgggcgtctccgtccgtttcgtctctgtagtcttcccagtccgaggcggggccgacacggccttcgaagacggagggggtttgtcgccgcgaggtagtaggcactggcccctgggtacatactactggggtgggacggacttccgacctgtgccttcgaattcgtttcctttagaccggcgagacgacccttcatactacacataaactagttaggggtcccttttcggaaagcgagatttcacctcaactaacgcatgaagcttttccatccgctgtgtagggacctgggattactaaaactgaagtgccattgaccctctccctcggggagggccgctctcgtctttggtggattcttcgggtttagagggtttcgaggtccttgaccgtctccggcccctgcggggtttccctcgccgtggtgctctgggttccgccggtgcagtctcccacacgtccacttttcccaggacctcttttcaggacccttcgaggaacagttctacggaaaagtttgaagcggtcccccgttccgactccccccaccccggtggtgtaggtgggtccagtaccactagtttgcggggccgtccttcgcttttcgactccggctgggagtccggtaagggttctttgccccggctttcggcccctcacaccaccgtcggcgacggcggctccggtttttctttcggcacttcctcagaagataggctagacacgtcctctggcatgaggggtagttcttcgcgttctgggccctctgccagtcgtagctccagttccttcaccacttcggggacgaccacaggtgggagccactcttctcgccctttcctgacttctggacattctcgggacccgccttttcgttcctctcgtcggggttccccgcgtcgtcgtcgcggaggagtggggggttcttcctcgtggtggtggtagtggtggtgagtctcaggggtttccgggggcacggtgacgagggtggggacgggggtggaggtggactcgggctctcgaggctcctggggtggtcggggggactcggggtcctgaactcgtcgtcgcagacgtttctcctcttctacgggtctcctccgagtgacctctcgctgccgacggggttcctcggtcgattctgagtcgggcgccaacggtggcggcggtgccggcgtcttttcatgtttgtggctcccctccctctcgcgtttctgtaacaaagtaggaggtacggttccggtttgtctctcctcggacacctgtcggcctgcgggcactggctctctcaatcgagatctcccgggaagcttgtttttgagtagagtcttctcctagaccagctgatctcgagcgactagtcggagtgttcttatttccgtcgacaacagagaagtcttcatcgaaacgtgaaaagatttgatccttatagtggtcctgacaatgagttacacacccatggctttcgtgactatataaatttttgttttccacattggataaataatatatttctcaaacggaatatttaaatgtatttttacaggcaaacacagaaaacaacatttttag